(19) United States
(12) Patent Application Publication
(10) Pub. No.: US 2022/0291060 A1
(43) Pub. Date: Sep. 15, 2022

(54) Elastomeric Sensor

(71) Applicant: Monash University, Clayton Victoria (AU)

(72) Inventors: Wenlong CHENG, Clayton Victoria (AU); Shu GONG, Clayton Victoria (AU); Lim Wei YAP, Clayton Victoria (AU)

(21) Appl. No.: 17/634,845

(22) PCT Filed: Aug. 12, 2020
(86) PCT No.: PCT/AU2020/050840

§ 371 Date: Feb. 11, 2022

(30) Foreign Application Priority Data
Aug. 12, 2019 (AU) 2019902903

(51) Int. Cl. G01L 1/22; G01K 7/22; G01H 11/06

(52) U.S. Cl. CPC G01L 1/2287; G01K 7/22; G01H 11/06; A61B 5/02444

(57) ABSTRACT

This disclosure relates to sensors comprising an elastomeric body incorporating a plurality of discrete electrical conductors such that an electrically conductive path can be formed within the elastomeric body via conduction between neighbouring conductors, and the elastomeric body includes at least one slit passing between neighbouring conductors. These sensors may be included in circuit structures, decals and vibration sensors. Also disclosed are methods of preparing the sensors, circuit structures and decals, and methods of using the sensors, circuit structures and decals.

This application claims priority to Australian provisional patent application no. 2019902903 filed on 12 Aug. 2019, and the entire contents of which are hereby incorporated by reference.

### FIELD OF THE INVENTION

The present invention relates to an elastomeric sensor and methods for forming the same.

### BACKGROUND OF THE INVENTION

Stretchable electronics (elastronics) is an emerging field that has increasing interest for applications in advanced biointegrated systems as well as the potential to integrate with stretchable optoelectronics to produce sophisticated soft robotics and displays. As core components, stretchable conductors (sensors/electrodes) provide the basic elements in these stretchable optoelectronic biointegrations. A requirement of this field is that the electronics are highly flexible to survive the mechanical deformation of the malleable host materials such as textiles, artificial skins, and soft biological parts.

Unlike traditional rigid electronic systems, it is crucial to design the interface of the different component materials to obtain highly stretchable electronics. This is because the intrinsic material mismatch between the conductor and polymer component materials often causes the interface to fail under mechanical deformation. For example, a problem that can arise with stretchable electronics comprising a polymer substrate layer with electrical conductors lying along the surface of the polymer substrate is debonding and/or delamination of the conductors from the polymer substrate. This may occur as a result of surface shear forces, or stretching and/or torsional forces applied to the polymer substrate.

Another important consideration for stretchable electronic sensors is that while they may be highly conductive, they generally have low strain sensitivity. Efforts have been made to increase sensitivity, for example by generating bulk channel cracks on rigid metal films. However such systems only function within a very small strain range (<10%). Thus, a key challenge is to provide a stretchable electronic sensor that is highly stretchable and strain sensitive.

Further still, it can be desirable to integrate multiple active materials within a single stretchable electronic system to realise multifunctional modalities. However, this is often achieved at the expense of device thickness and mechanical deformability.

The present invention seeks to address at least one of the aforementioned problems.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In a first aspect, the present invention provides a sensor comprising an elastomeric body, said elastomeric body incorporating a plurality of discrete electrical conductors therein such that an electrically conductive path can be formed within the elastomeric body via conduction between neighbouring conductors, said elastomeric body including at least one slit passing between neighbouring conductors.

Advantageously, deformation of the elastomeric body changes a separation between neighbouring conductors having a slit therebetween to thereby change the conductivity of the electrically conductive path within the elastomeric body.

Deformation of the elastomeric body may change a geometric property of the slit which thereby changes a separation between neighbouring conductors separated by the slit to change the electrical conductivity of the electrically conductive path.

In an embodiment, the elastomeric body can be formed from any suitable viscoelastic polymer. The elastomeric body may exhibit elastic properties (such as stretches, bends, twists and compression) when mechanically deformed. Preferably, the elastomeric body can be formed from one or more of: polydimethylsiloxane (PDMS), rubbers (such as a recycled rubber available from Eco-flex), silicones, polyurethanes, and combinations thereof. In some embodiments, the elastomeric body comprises PDMS. In a preferred embodiment, the elastomeric body is PDMS.

In an embodiment, the thickness of the elastomeric body from its top to bottom surface is between about 1 μm to about 10 cm, preferably about 10 μm to about 500 μm, more preferably about 10 μm, about 100 μm, about 200 μm, or about 500 μm.

The discrete electrical conductors can be formed from any one or more of: metals, semiconductors, reduced graphite oxide, and combinations thereof. Suitable metals include, but are not limited to, Ag, Au, Cu, Ir, Nb, Os, Pd, Pt, Re, Rh, Ru, Ta, Ti and mixtures thereof. Preferred metals include Ag, Au, Cu, Pd, and Pt, more preferably Au and Ag. Preferably each of the electrical conductors within the elastomeric body are of substantially the same composition.

In some embodiments, the discrete electrical conductors may be provided in the form of sheets, wires, rods, spheres, or combinations thereof. Preferably, the discrete electrical conductors are provided in the form of nanosheets, nanowires, nanorods, nanospheres, or combinations thereof. Preferably each of the discrete electrical conductors within the elastomeric body are of substantially the same structure. In a preferred embodiment, the discrete electrical conductors within the elastomeric body are nanowires.

In a particularly preferred embodiment, the discrete electrical conductors are gold nanowires.

Neighbouring conductors may form an electrically conductive path through the elastomeric body when electrical charge carriers can move between neighbouring conductors with the application of voltage. Generally speaking this may occur when the neighbouring conductors contact one another at one or more points or are separated by less than 1 nm.

The discrete electrical conductors may be arranged in subgroups containing at least one discrete electrical conductor wherein a conductive path is formed through the subgroup, wherein each subgroup is separated from a neighbouring subgroup by a slit such that electrical conduction between neighbouring conductors in adjacent subgroups may occur across the slit. In such embodiments deformation of the elastomeric body may change a geometric property of the slit which thereby changes a separation between said adjacent subgroups to change the electrical conductivity between adjacent subgroups. In an embodiment, the elastomeric body incorporating discrete electrical conductors has a density of discrete electrical conductors from about 60 μm⁻² to 1.10×10⁴ μm².

By way of example, discrete electrical conductors in the form of nanowires may be arranged in the form of a standing nanowire array, wherein the nanowires adopt a substantially vertical orientation. In some embodiments, the standing gold nanowires may be referred to as vertically-aligned gold nanowires. In one embodiment, the nanowires may be substantially parallel to one another. In another embodiment, the nanowires may be arranged in subgroups wherein a nanowire within a subgroup contacts one or more nanowires within the subgroup at one or more points. In one form of this embodiment, the nanowire subgroup forms a 3D array where a conductive path is formed through the subgroup.

A discrete electrical conductor may be completely incorporated in the elastomeric body, wherein the entirety of the electrical conductor is contained within the elastomeric body. A discrete electrical conductor may be partially incorporated in the elastomeric body wherein a portion of the electrical conductor is contained within the elastomeric body.

A slit will be understood to refer to a discontinuity in the elastomeric body that extends at least partly between at least two neighbouring conductors. Such slits may be of any shape or depth. In a preferred form, when the elastomeric body is unstained, electrical conduction between neighbouring conductors that are separated by the slit may occur across the slit. A slit as defined herein may be formed by any suitable method including:
mechanical methods such as cutting (e.g. with a blade), breaking, cracking, cleaving, splitting, engraving, or stamping;
application of energy such as EM radiation or plasma to remove material;
a chemical process such as etching, or removal of sacrificial material; or
moulding or forming the elastomeric body with slits.

In some embodiments, the elastomeric body has at least one surface. The slit may extend from the surface into the elastomeric body. Preferably, the slit extends through the elastomeric body such that it passes completely or partially between at least some neighbouring conductors. In a preferred embodiment, the electrical conductors are incorporated in a region of the body adjacent to said surface; and said slit extends from said surface into said body. Preferably the slit extends from the surface of the elastomeric body such that it passes completely or partially between at least some neighbouring conductors.

In an embodiment, a slit extends into the elastomeric body from a surface thereof, the slit including two faces opposed to each other, which reach the surface at shoulder portions thereof, and extend towards a valley between them. The geometric property of the slit that is changed by deformation of the elastomeric body can include any one or more of the following:
a fraction of the slit faces that are in contact;
a separation between the slit faces at a point;
an average or other representative separation between slit faces in total, over a region, at a point, along a line.

A slit can be elongate, and may be of substantially uniform depth from the surface. A slit can be substantially linear, curved, random, meandering, smooth or jagged.

In an embodiment, the slit may have an average depth of about 100 nm to about 1.5 μm, preferably about 500 nm to about 1.2 μm, more preferably about 500 nm, about 600 nm, about 700 nm, about 800 nm, about 900 nm, about 1 μm, about 1.1 μm or about 1.2 μm. The average depth of the slit may be about 0.05% to about 15% the thickness of the elastomeric layer, preferably about 0.25% to about 12%, more preferably about 0.05%, about 0.25%, about 0.3%, about 0.35%, about 0.4%, about 0.45%, about 0.5%, about 0.55%, about 0.6%, about 1%, about 5%, about 6%, about 7%, about 8%, about 9%, about 10%, about 11% or about 12%.

Preferably, the elastomeric body comprises a plurality of slits.

In an embodiment, the plurality of slits may have an average spacing of about 20 μm to about 800 μm, preferably about 50 μm, about 250 μm, or about 600 μm.

In an embodiment, the plurality of slits may exhibit an orientation selected from: unidirectional, bidirectional or multidirectional. Slit orientation refers to the alignment of slits comprising the plurality of slits. A plurality of slits that exhibits a unidirectional orientation comprises slits that are aligned substantially parallel to one another. A plurality of slits that exhibits a bidirectional orientation comprises a first and second plurality of slits, wherein the first plurality of slits comprises slits substantially parallel to one another, the second plurality of slits comprises slits substantially parallel to one another, wherein the first and second plurality of slits are not parallel to one another. In some embodiments the discrete electrical conductors are arranged to form at least one track in the elastomeric body. Said tracks provide a conductive path through the elastomeric body. In such cases the one or more slits can be arranged transverse to the track. The track can be any shape, but will in most cases be elongate, with the slits traversing across a short dimension of the track. A plurality of slits may exhibit a multidirectional orientation, for example the slits may be aligned transverse to the electrically conductive track in a circular or spiral track path.

In an embodiment, the sensor has a gauge factor of about 10 to about 1400. As used herein, gauge factor (GF) is defined as the normalized electrical resistance variation value ΔR/(R₀×_{ε}), where ΔR is the change in electrical resistance, R₀ is the initial electrical resistance, ε is the tensile strain.

In an embodiment, the sensor has a stretchability limit of about 5% to about 80%. As used herein, stretchability limit is defined as the critical strain at which the sensor loses conductivity.

In another aspect, the present invention provides a circuit structure including one or more sensors according to any embodiment of the first aspect of the invention. In a preferred form the one or more sensors share a common elastomeric body.

The circuit structure can further include at least one of:
one or more further sensors; and
one or more conductive tracks for electrically connecting other circuit elements.

Preferably the further sensors or conductive tracks comprise an elastomeric body, said elastomeric body incorporating a plurality of discrete electrical conductors therein such that an electrically conductive path can be formed within the elastomeric body via conduction between neighbouring conductors. Preferably, the elastomeric body forming the further sensors and/or conductive tracks does not include a slit. Preferably the one or more sensors, and any further sensor(s); and/or conductive tracks share a common elastomeric body.

In an embodiment, the circuit structure may comprise two or more sensors according to any embodiment of the first aspect of the invention. In another embodiment, the circuit structure may comprise one or more sensors according to any embodiment of the first aspect of the invention and one or more further sensors.

The circuit structure may comprise a partial circuit configured to be completed by the addition of one or more further circuit components. For example the further components could include a power source. The further components could include, without limitation, a communications module, control system, measurement or readout system etc.

In a preferred embodiment the circuit structure can comprise a unitary elastomeric body comprising an elastomeric layer incorporating a plurality of discrete electrical conductors therein such that an electrically conductive path defining the circuit structure (e.g. one or more sensors, further sensors or conductive tracks) can be formed within the elastomeric body via conduction between neighbouring conductors. Said one or more sensors may be defined in one or more corresponding portions of said electrically conductive path by providing at least one slit in the elastomeric body at said corresponding portion or portions.

The circuit structure can comprise a continuous sheet-like elastomeric layer with the circuit structure defined by a pattern or arrangement or discrete electrical conductors incorporated therein; or a shaped elastomeric layer having a shape defined by a pattern or arrangement of discrete electrical conductors incorporated therein.

In another aspect, the present invention provides a decal comprising a first layer including at least one sensor according to the first aspect of the invention and a substrate. The at least one sensor of the first layer can comprise a circuit structure in accordance with an embodiment of the second aspect of the present invention.

In some embodiments the first layer can comprise a unitary elastomeric layer providing the elastomeric body of said at least one sensor.

The substrate can be formed from a polymer, paper, fabric, rigid substrate, or combination thereof. Preferably, the substrate is formed from polyvinyl alcohol (PVA).

The decal can comprise one or more additional layers, including but not limited to: an adhesive layer, a release layer, a printed layer, and a protective layer.

In another aspect, the present invention provides a vibration sensor comprising:
at least one sensor according to an embodiment of the first aspect of the present invention; and
a carrier to which the at least one sensor is mounted, such that vibrations to be sensed cause deformation of the sensor.

The carrier preferably comprises a frame including at least one support member defining a void, wherein the sensor is carried on the at least one support member and extends across the void. In a preferred embodiment a portion of the sensor extends across the void without touching the carrier within said void such that the portion of the sensor is free to move in response to vibrations. The sensed vibrations can propagate through any medium, including air or water.

The frame can include a pair of support members, wherein a first support member of said pair is located on one side of the void, and the second support member of said pair is located on the other side of the void, such that a sensor carried by the pair of support members extends across the void.

In embodiments including a plurality of sensors, each sensor can be:
formed in the same elastomeric body as at least one other sensor;
formed in an elastomeric body with all other sensors;
formed on an elastomeric body with no other sensor.

The void may be of any shape including, but not limited to, fully open, a blind cavity, a cavity with one or more openings therein, a channel with one or more open ends.

The elastomeric body can comprise a layer of elastomeric material. Preferably the elastomeric layer has a thickness of between about 10 μm and about 200 μm.

In one form the carrier is of unitary construction.

In a preferred form the frame is an elongate structure having a pair of laterally spaced apart support members defining an elongate void therebetween, said pair of support members carrying a plurality of sensors therebetween said plurality of sensors being spaced along the support members. In a some embodiments the pair of support members are arranged such that the void therebetween has a varying width between the support members, such that at least one of the sensors is of a different length to another of said plurality of said sensors. In a preferred embodiment a separation between the pair of support members widens from one end to another such that the void therebetween has an increasing width from said one end to the other, such that the sensors increase in length from one to the next along said support members. Preferably the length of the sensors is arranged such that each sensor has a different resonance frequency. Most preferably the resonance frequencies of the sensors lie in the range of 40 Hz to 3000 Hz. In some embodiments the sensor(s) can be adapted to have a resonance frequency higher than this range. For example for a sensor adapted to sense vibrations in water the resonance frequency can be up to 1200 kHz, or other frequency used in sonar sensing.

In yet another aspect, the present invention provides a method of preparing a sensor, comprising:
providing an elastomeric body, said elastomeric body incorporating a plurality of discrete electrical conductors therein such that an electrically conductive path can be formed within the elastomeric body via conduction between neighbouring conductors; and
forming at least one slit passing between neighbouring conductors.

The slit may be formed by any suitable method including:
mechanical methods such as cutting (e.g. with a blade), breaking, cracking, cleaving, splitting, engraving, or stamping;
application of energy such as EM radiation or plasma to remove material;
a chemical process such as etching, or removal of sacrificial material; or
moulding or forming the elastomeric body with slits.

Preferably, the slit may be formed by mechanical methods, more preferably by cracking. In embodiments wherein the slits are formed by cracking, the slits may be referred to as cracks. In a preferred embodiment, the method comprises forming a slit traversing across a short dimension of the conductive track by applying a strain along the direction of the track.

In a preferred embodiment, the method comprises forming a plurality of slits.

The elastomeric body incorporating a plurality of discrete electrical conductors therein may be prepared by any suitable method. In one embodiment, the discrete electrical conductors may be formed and then incorporated within an elastomeric body, for example by spin-coating and curing a polymer substrate. In another embodiment, the discrete electrical conductors may be formed on a semi-cured polymer substrate, such that following curing, the discrete electrical conductors are incorporated within the cured polymer substrate.

In some embodiments the method includes; providing a mask overlying the elastomeric body to define a region in which the said at least one slit is to be formed.

The method can include forming a rigid layer overlying the elastomeric body; and applying a strain to said elastomeric body to crack said rigid layer, whereby said cracks propagate into said elastomeric body to form at least one slit therein.

In some embodiments the method can include, forming the rigid layer on the elastomeric body through said mask.

The rigid layer may be formed from any suitable material that forms a slit under strain. Preferably, the rigid layer can be formed from one or more of: a metal, reduced graphite oxide, or a combination thereof. Preferably, the rigid layer is formed from a metal. Suitable metals include, but are not limited to, Ag, Au, Pt, Cu and Ti, preferably Ag and Au.

Preferably the thickness of the rigid layer from its top to bottom surface is between about 100 nm to about 500 nm, more preferably about 120 nm, about 250 nm or about 400 nm.

Preferably, the strain applied to the elastomeric body is between about 10% to about 80%, more preferably about 15%, about 30%, about 45%, about 60% or about 75%.

The method can further include removing said rigid layer. The rigid layer may be removed by any suitable method including, mechanical or chemical removal.

In yet another aspect, the present invention provides a method of preparing a circuit structure comprising one or more sensors according to any embodiment of the first aspect of the invention, comprising:
providing an elastomeric body, said elastomeric body incorporating a plurality of discrete electrical conductors therein such that an electrically conductive path is formed within the elastomeric body via conduction between neighbouring conductors; and
generating the one or more sensors by forming at least one slit passing between neighbouring conductors in a region of the elastomeric body.

In a preferred embodiment, the method comprises forming a plurality of slits in the region.

Preferably, the elastomeric body includes a further region that does not include a plurality of slits. The further region may form a conduction track for electrically connecting circuit elements.

Also disclosed herein is an elastomeric body, said elastomeric body incorporating a plurality of discrete electrical conductors therein such that an electrically conductive path can be formed within the elastomeric body via conduction between neighbouring conductors, said elastomeric body including at least one slit passing between neighbouring conductors. The elastomeric body may be any of the elastomeric bodies described herein in the context of a sensor and/or a circuit and/or a decal.

As used herein, except where the context requires otherwise, the term “comprise” and variations of the term, such as “comprising”, “comprises” and “comprised”, are not intended to exclude further additives, components, integers or steps.

As used herein and in the appended claims, the singular forms “a”, “an” and “the” include plural reference unless the context clearly dictates otherwise. Thus, for example, a reference to “a slit” or “at least one slit” may include one or more slits (eg a plurality of slits).

The term “and/or” can mean “and” or “or”.

The term “(s)” following a noun contemplates the singular or plural form, or both.

Various features of the invention are described with reference to a certain value, or range of values. These values are intended to relate to the results of the various appropriate measurement techniques, and therefore should be interpreted as including a margin of error inherent in any particular measurement technique. Some of the values referred to herein are denoted by the term “about” to at least in part account for this variability. The term “about”, when used to describe a value, may mean an amount within ±10%, ±5%, ±1% or ±0.1% of that value.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1 is a schematic representation of steps of fabrication process of standing gold nanowires (V-AuNWs) incorporated in PDMS substrate and shows: (1) Silicon wafer with PMMA layer was modified with (3-Aminopropyl)triethoxysilane (APTES); (2) gold seeds deposition; (3) In-situ growth of V-AuNWs; (4) Spin-coating and curing of PDMS; and (5) lifting-off PDMS with V-AuNWs.

FIG. 2 is a cross-sectional image from a scanning electron microscopy (SEM), of V-AuNWs incorporated in PDMS substrate.

FIG. 3a, b are top-view SEM images of V-AuNWs/PDMS before applying uniaxial tensile strain, and (c), (d) are Top-view SEM images of V-AuNWs/PDMS after applying 20% uniaxial tensile strain.

FIG. 4(a-c) are optical images of V-AuNWs/PDMS thin film; at initial state (a); under 80% uniaxial tensile strain (b); and after releasing from tensile strain (c) at the same spot.

FIG. 5a is a schematic representation of a fabrication process for generating localized cracks within a V-AuNW/PDMS thin film. 5(b) is an Optical image of a V-AuNW/PDMS thin film including regions with and without cracks. 5(c) plots the resistance changes of cracked area and non-cracked area from 5(b) under a stretching-releasing cycle with a uniaxial tensile strain of 0%-60%-0%. 5(d-f) are optical images of a cracked film: before stretching 5(d); during stretching 5(e); and after releasing 5(f) from a tensile strain of 60%. Scale bar 200 μm.

FIG. 6 is a top view atomic force microscopy (AFM) image of a typical crack. Insets are height profilometry data of the crack.

FIG. 7a plots the resistance response of a typical non-cracked V-AuNWs/PDMS thin film under a uniaxial tensile strain of 0%-60%, sweep rate 1 mm/min. 7(b) shows the resistance response of the film under repeated cycles of strain from 0%-2%-0% to 0%-20%-0%.

FIG. 8a, b are cross-section SEM images of a typical V-AuNWs/PDMS crack before (a) and under (b) a tensile strain of 20%. Scale bar 1 μm.

FIG. 9a plots the current changes of a cracked V-AuNWs/PDMS during a durability test for 1,000 cycles with repeated 0-60%-0% strain. A constant voltage of 0.1 V was applied to the sample. FIG. 9b, 9c show enlarged views of the first 4 cycles, and the last 4 cycles of FIG. 9a. FIG. 9d plots the current changes of a cracked gold thin film (˜50 nm) during a durability test for 10 cycles with repeated 0-60%-0% strain.

FIG. 10a, b show optical images of cracked gold thin film before (and after 10 repeated cycles under 0-60%-0 strain. FIG. 10c, 10d show optical images of cracked V-AuNWs film before (a) and after (b) 1,000 repeated cycles under 0-60%-0 strain.

FIG. 11a-e show height profiles and corresponding AFM images of V-AuNWs cracks generated with pre-strain level of 15% 11a, 30% 11b, 45% 11c, 60% 11d, and 75% 11e. FIG. 11f plots the relationship between pre-strain level and depth of cracks measured from AFM. Each measured depth was averaged from three samples.

FIG. 12 plots the relationship between sputtering coating time and metal thickness measured by AFM.

FIG. 13a-c shows height profiles and corresponding AFM images of V-AuNWs cracks generated with different thickness of Ag layer. 120 nm 13a, 250 nm 13b, and 400 nm 13c. FIG. 13d shows the relationship between thickness of Ag and depth of cracks measured from AFM. Each measured depth was averaged from three samples.

FIG. 14a shows optical images of V-AuNWs cracks generated with different thickness of Ag layer. FIG. 14b shows the relationship between thickness of Ag film and average density of cracks. Each result was averaged from three samples.

FIG. 15a shows optical images of V-AuNWs cracks generated with different pre-strain levels. FIG. 15b illustrates the relationship between pre-strain levels and average density of cracks. Each result was averaged from three samples.

FIG. 16 illustrates the sensing performance of V-AuNWs/PDMS thin films with programmable cracks. FIG. 16a is a mapping of gauge factors distribution of cracked V-AuNWs/PDMS thin film as a function of pre-strain level and metal thickness. FIG. 16b is a mapping of stretchability distribution of cracked V-AuNWs/PDMS thin film as a function of pre-strain level and metal deposition time. FIG. 16c plots the gauge factors of cracked V-AuNWs/PDMS thin film with length of cracked area of 0-2.5 mm. FIG. 16d plots the gauge factors of cracked V-AuNWs/PDMS thin film with width of cracked area of 0-2.0 mm. FIG. 16e plots the gauge factors of cracked V-AuNWs/PDMS thin film with different crack orientation. Tensile strains were applied along horizontal direction. FIG. 16f shows the resistance changes of a square-shaped cracked thin film with repeated tensile strain of 10%. The crack orientation is in parallel to y axis while the strain direction varied with an angle θ to x axis.

FIG. 17a plots the resistance changes of cracked V-AuNWs thin film with the increase of tensile strain. The GFs and stretchability limit could be tuned in the range of 10-1362 and 79%-8.8%, respectively. FIG. 17b shows an enlarged view in the strain range of 0-10%.

FIG. 18a-c show optical images of V-AuNWs/PDMS thin films after deposition a layer of silver with different shapes: 200×200 μm² square (a); 300×300 μm² square (b); three circular patterns with diameters of 100, 200 and 300 μm, respectively (c). FIG. 18d-f show corresponding optical images after generation of cracks and removal of silver from 18a-c, respectively. Scale bar: 200 μm.

FIG. 19a plots resistance changes of a V-AuNWs/PDMS strip in the strain range of 0-10% with programmable length of cracked area from 0-2.5 mm. FIG. 19b plots resistance changes of a V-AuNWs/PDMS strip in the strain range of 0-10% with programmable width of cracked area from 0.5-2.0 mm. FIG. 19c plots resistance changes of a V-AuNWs/PDMS strip in the strain range of 0-10% with programmable orientation of cracks.

FIG. 20a shows a schematic of the morphology changes of channel cracks (slits) with 0% strain (left), 20% x axis strain (middle) and 20% y axis strain (right). FIG. 20b shows the corresponding in-situ AFM images and height profiles of a sample with 0% strain (left), 20% x axis strain (middle) and 20% y axis strain (right). FIG. 20c shows the corresponding finite-element method modelling results of crack interfacial deformation with 0% strain (left), 20% x axis strain (middle) and 20% y axis strain (right).

FIG. 21a is a schematic of the strain components applied to the cracked E-skin. FIG. 21b plots resistance changes of the cracked E-skin with strain ranging from 0-10% at the strain direction 0°-90° to x axis.

FIG. 22a shows the resistance changes of a cracked bulk gold film with periodic strain in the range of 0.5%-10% along y-axis direction. FIG. 22b, 22c are optical images of the cracked bulk gold film before 22b and after 22c applying a periodic strain at 10% for 100 cycles. Scale bar: 200 μm. FIG. 22d shows the resistance changes of a cracked V-AuNWs/PDMS film with periodic strain in the range of 0.5%-10% along y-axis direction. FIG. 22e, 22f are optical images of the cracked V-AuNWs/PDMS film before FIG. 22e and after 22f applying a periodic strain at 10% for 100 cycles.

FIG. 23a is a schematic illustration of localized cracks on a decal-like V-AuNWs based ultrathin interconnects. FIG. 23b is a photograph of V-AuNWs/PDMS interconnects attached to skin as decals with both nanowire side on top and nanoparticle side on top. Scale bar 1 cm. FIG. 23c-d are photographs of V-AuNWs/PDMS interconnects (conductive tracks) when they are compressed (c) and stretched (d). Scale bar: 1 cm.

FIG. 24 illustrate various details of an integrated system 24a is a photograph of a spiral-shaped pulse sensor based on cracked V-AuNWs/PDMS thin film with BLE components. Scale bar: 2 cm. FIG. 24b shows a comparison of sensor response before and after crack generation. FIG. 24c is a schematic illustration of the location and morphologies of three crack sensors integrated in a “gold E-skin” pattern. FIG. 24d is a photograph of the tattoo-like “gold E-skin” circuit attached onto dorsal aspect of hand as a decal. FIG. 24e-j are frames from video clips of the response of three sensors under both x-axis and y-axis strain.

FIG. 25a is an optical image of a V-AuNWs cracked spiral-shaped pulse sensor. FIG. 25b, 25c are optical images of the spiral sensor before 25b and after 25c cracks generation. Scale bar: 200 μm.

FIG. 26 shows the calculated quality factors for each pulse waveform in a duration of 8.5 s measured from the pulse sensor with channel cracks FIG. 26a and without channel cracks FIG. 26b.

FIGS. 27a and 27b are simulations of blood vessel before (a) and after (b) inflation. FIG. 27c shows a typical wrist pulse wave measured from the pulse sensor with channel cracks. FIG. 27d shows the second derivative with five distinct characteristic points. The pulse was obtained from a 30-year-old man.

FIG. 28a shows an optical image of a bi-directional cracked V-AuNWs thin film. Scale bar 500 μm. FIG. 28b is a magnified optical image of 28a. Scale bar: 200 μm.

FIG. 29 shows a multifunctional sensory system based entirely on V-AuNWs with programmed cracks. FIG. 29a is a photograph of the multifunctional sensory system attached on human skin. Scale bar: 1 cm. FIG. 29b plots the resistance responses of strain sensor arrays (S1-S4) when a cyclic strain of 0-5% is applied. FIG. 29c plots the resistance responses of pressure sensor arrays (P1-P4) when a cyclic pressure of 0-50 kPa is applied. FIG. 29d plots the resistance responses of strain direction sensor arrays (D1-D4) when a strain of 5% is applied with four strain directions (as see in scheme below). FIG. 29e plots the resistance changes of a temperature sensor in the range of 20-55° C. FIG. 29f plots the current changes of a glucose sensor with the addition of glucose solution in various amount. Insert is the sensitivity of the glucose sensor in the glucose concentration of 0-2.5 mM. FIG. 29g plots the current changes of a lactate sensor with the addition of lactate solution in various amount. Insert is the sensitivity of the lactate sensor in the lactate concentration of 0-30 mM.

FIG. 30a is an optical image of a V-AuNWs-based temperature sensor. Scale bar 1 cm. FIG. 30b plots the dynamic resistance changes of the temperature sensor under cyclic heat on and heat off in the temperature range of 25° C.-45° C.

FIG. 31a illustrates steps in a fabrication process for creating a vibration sensor, implemented as an acoustic sensor in an embodiment of the present invention. FIG. 31b shows a scanning electron microscopy image of the cracked area of a sensor used in an exemplary embodiment. FIG. 31c illustrates a plot of current changes of an exemplary sensor with dynamic tensile strain ranging from 0.2%-50% over 10 stretching-releasing cycles at each strain level.

FIG. 32a-c are schematic illustrations of the experimental set-ups for acoustic measurement from a sensor without cracks (a), a sensor according to an embodiment of the present invention with uniform cracks (b), and a sensor according to an embodiment of the present invention sensor with localized cracks (c). FIG. 32d-f show a comparison of vibrometer output and sensor output from the sensors of FIG. 32a-c respectively for application of a chirp signal of varying frequency of 40-3000 Hz over 2 s. FIG. 32g shows an enlarged view of resistance changes with time in three different time zones indicated in FIG. 32f.

FIG. 33 illustrates the response to a sensor using a 300 um PDMS layer, and an equivalent sensor with a 10 um PDMS layer.

FIG. 34a is a plot of the variation in resistance changes of an exemplary sensor as a function of sound frequency from 0-1000 Hz. FIG. 34b is a plot of resistance outputs of an exemplary sensor under sound with a constant frequency of 400 Hz at SPL of 90 dB. FIG. 34c shows the variation in resistance changes of an exemplary sensor under sound with a constant frequency of 80 Hz at SPL from 70-95 dB. FIG. 34d is a schematic of the experiment set-up of the music detection used to obtain the data of figures FIG. 34a-c, and e-k. FIG. 34e shows the sensor output (curve) and the STFT analysis (background) in response to musical notes at different pitches. FIGS. 34f and 34g show the exemplary sensor output and waveform, compared to the conventional microphone output waveform in FIG. 34h in response to a piece of music. FIG. 34i-k show enlarged sections of the potions of the curves highlighted in dashed boxes in FIG. 34f-h.

FIG. 35 shows the output of a sensor in response to discrete musical notes and spectral analysis of the output by FFT.

FIG. 36 shows the normalised output of a sensor in response to music notes for an exemplary audio sensor and commercial microphone.

FIG. 37a is a conceptual schematic of human ear with an uncoiled cochlea. FIG. 37b is a schematic illustration of a vibration sensor implemented to provide an artificial basilar membrane including 8 sensor strips with localized cracks in the center. FIG. 37c is a photograph of a soft nanowire-based artificial basilar membrane made in accordance with FIG. 37b (scale bar 1 cm). FIG. 37d is an optical image showing the cracked area of a sensor of the device of FIG. 37c (Scale bar: 200 μm).

FIG. 38 schematically illustrates steps in a fabrication process for a nanowire-based soft artificial basilar membrane.

FIG. 39a illustrates resistance changes of “sensor 7” in an exemplary ABM, upon application of a chirp sound at various frequency ranges. FIG. 39b plots the resistance changes of “sensor 3” of the ABM upon application of a chirp sound with various time ranges.

FIG. 40a is a schematic illustration of the experiment set-up for testing the sensor of FIG. 37b. FIG. 40b-d illustrate: sensor resistance response (b), sensor waveform (c), and short-time Fourier transform signals of a sensor strip under a chirp signal varying from 40-3000 Hz over for 2 s. FIG. 40e illustrates the acoustic-to-mechanical transfer function for all sensor strips of the exemplary ABM in the frequency range from 40-3000 Hz. FIG. 40f illustrates the sensitivity for all sensor strips in the frequency range from 40-3000 Hz. FIG. 40g plots the resonance frequencies of the sensors of the ABM determined from vibrometer output and sensor output. FIG. 40h shows the dependence of the sensor output of the exemplary ABM on the sound pressure level when applying a pure tone at the resonance frequency of each sensor.

FIG. 41 illustrates the output of 4 sensors with application of different frequency sounds.

### DETAILED DESCRIPTION OF THE EMBODIMENTS

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognise many methods and materials similar or equivalent to those described herein, which could be used in the practice of the present invention. The present invention is in no way limited to the methods and materials described.

It will be understood that the invention disclosed and defined in this specification extends to all alternative combinations of two or more of the individual features mentioned or evident from the text. All of these different combinations constitute various alternative aspects of the invention.

V-AuNWs/PDMS Thin Film

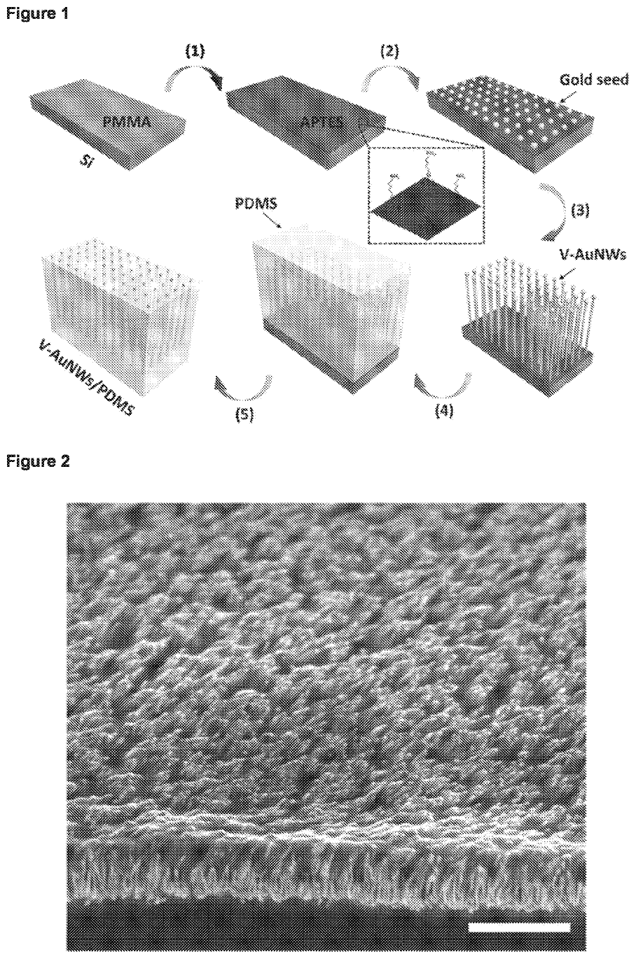

A process for the preparation of a sensor comprising an elastomeric body, wherein the elastomeric body incorporates a plurality of discrete electrical conductors therein such that an electrically conductive path can be formed within the elastomeric body via conduction between neighbouring conductors is depicted in FIG. 1. As shown herein, the elastomeric body is formed from PDMS, and the discrete electrical conductors are formed from gold nanowires (AuNWs). The AuNWs form a standing nanowire array, wherein an electrically conductive path can be formed within the PDMS elastomeric body via conduction between neighbouring AuNWs.

As shown in FIG. 1, the AuNW array may be prepared using a sacrificial PMMA layer pre-treated with APTES to functionalise the surface of the PMMA such that Au seed nanoparticles adsorb to the surface of the PMMA. The surface may then be exposed to a growth solution to grow AuNWs. In this embodiment, the AuNWs are grown on the sacrificial PMMA substrate and then incorporated in a PDMS elastomeric body via spin coating and curing of PDMS. The sacrificial PMMA substrate may then be removed to provide the AuNWs/PDMS thin film.

Alternatively, the AuNW array may be grown on a semi-cured substrate, such that following curing the cured substrate incorporates the AuNWs. For example, AuNWs may be grown on semi-cured PDMS as described in Australian provisional application 2019901857 and in Zhu B. et al, Adv. Electron. Mater. (2019) 5, 1800509.

The incorporation of the AuNWs within the PDMS elastomeric body was confirmed by cross-sectional scanning electron microscope (SEM) image (FIG. 2).

v-AuNWs/PDMS Thin Film Including Slits

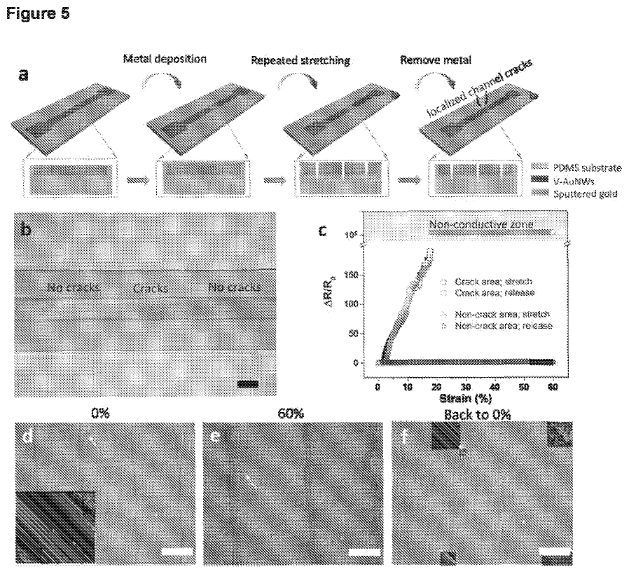

A process for the preparation of a sensor comprising an elastomeric body, wherein the elastomeric body incorporates a plurality of discrete electrical conductors therein such that an electrically conductive path can be formed within the elastomeric body via conduction between neighbouring conductors, said elastomeric body including at least one slit passing between neighbouring conductors is depicted in FIG. 5. As shown herein, the elastomeric body includes a plurality of slits in the form of cracks. The cracks were generated by depositing rigid materials on top of the AuNWs/PDMS elastomeric body, followed by repeated tensile stretching, which led to cracks forming in the transverse direction to the extension strain applied. Any suitable rigid material may be used. Preferably, the rigid material is a thin film of metal (gold or silver) sputter coated onto a specific location through shadow masks (FIG. 5).

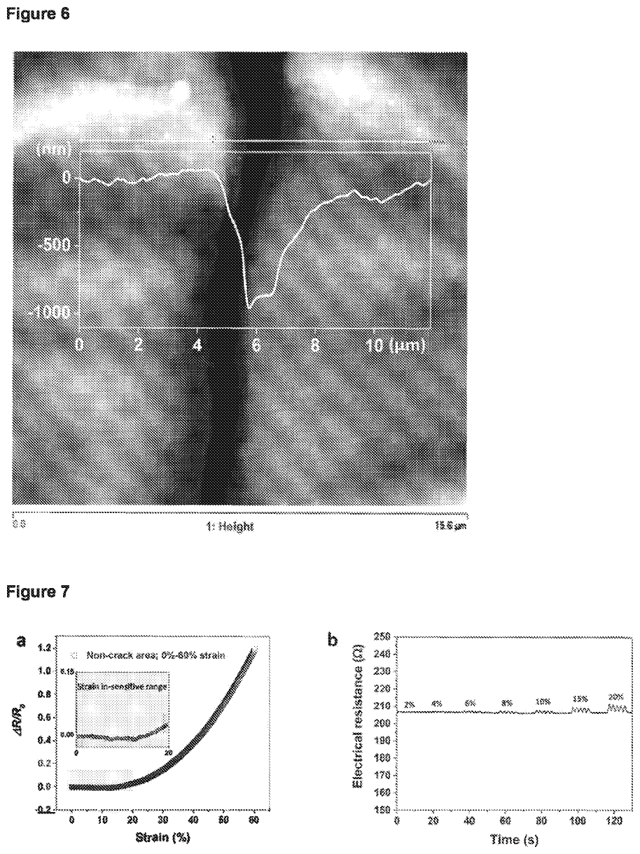

The repeated tensile stretching did not induce any cracks on the locations without sputtered metal deposition. The sputtered metal thin film may be removed by either adhesive tape (gold) or dissolved by hydrogen peroxide/ammonium hydroxide solution (silver), leaving localized parallel channel crack replicas (FIG. 5b). Atomic force microscopy (AFM) characterization showed cracks propagating from the top surface of the PDMS elastomeric body into the PDMS elastomeric body between gold nanowires (FIG. 6).

Characterisation of v-AuNWs/PDMS Thin Film Including Slits

The cracked area of the AuNWs/PDMS thin film showed dramatically different electrical responses to tensile strains compared to the corresponding non-cracked area.

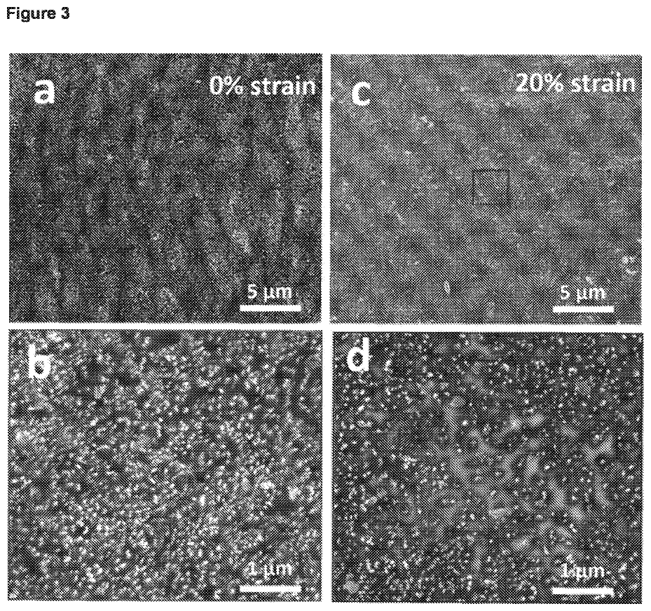

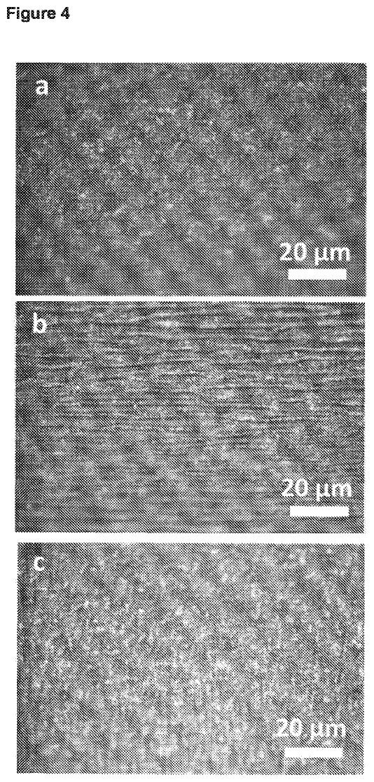

In particular, the inventors observed that an AuNWs/PDMS thin film that does not include slits was strain-insensitive. As shown in FIG. 3, AuNWs/PDMS thin films were resistant to forming cracks under strain. No evident cracks were observed up to 80% strain (FIG. 4).

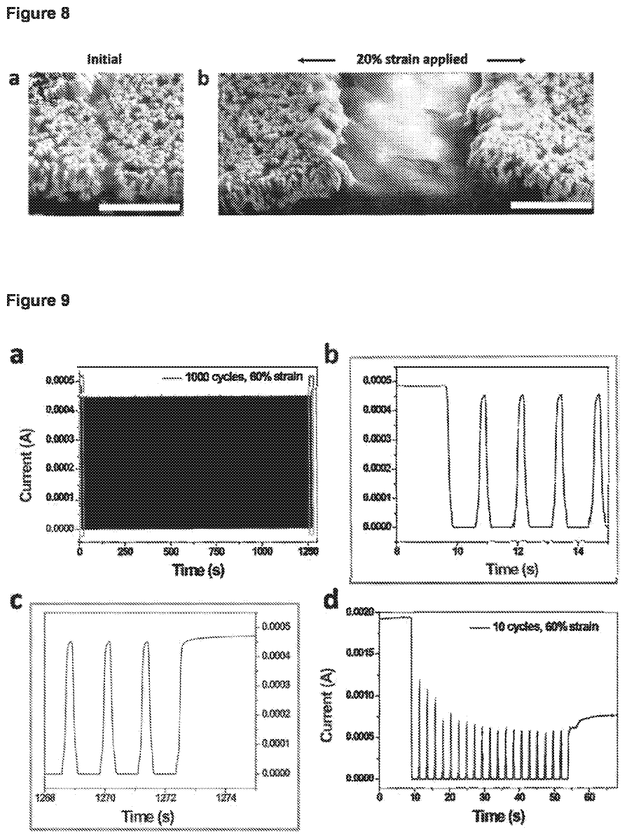

In a typical experiment with the tested electrode size of 2×0.5 mm² over a finite range of strain (FIG. 5c), the non-cracked area was almost insensitive to strain with an average of gauge factor (GF) of only ˜0.17 in the strain range of 0-20% (FIG. 7); whereas, the cracked area was very sensitive to strains with significant changes in conductivity, giving rise to a GF of ˜1035 within 20% strain. The reduction in conductivity was attributed to crack enlargement (FIG. 8).

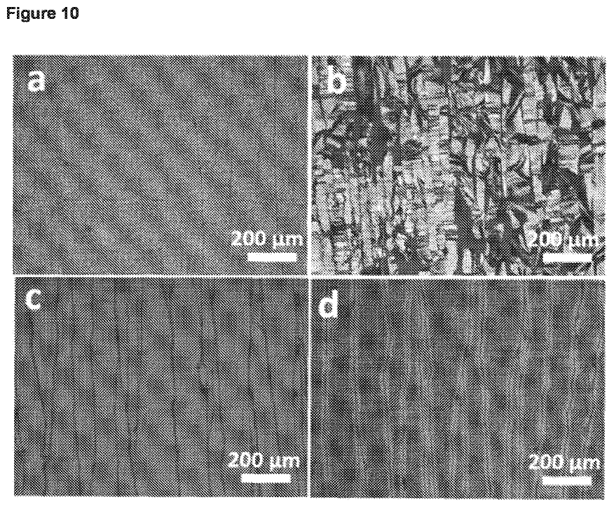

Surprisingly, crack repair was observed upon strain release (FIG. 5d-f), leading to full recovery of the electrical profile as shown by circles in FIG. 5c. The cracked area lost its conductivity completely up to a tensile strain of ˜18.4%, however, its electrical conductance could be fully recovered from an extensive strain of 60%. This was further confirmed by a dynamic durability test of 1,000 cycles with repeated strain of 60% (FIG. 9a-c). The high durability under high repeated strain test is in contrast with previous reported sputter-coated metallic bulk-cracking system, which could only allow a limited stretchability of <10%. In a control experiment, a cracked sputtered gold thin film lost the majority of its conductivity after only 10 cycles with the same repeated strain (FIG. 9d), due to severe delamination (FIGS. 10a and 10b). Such delamination was not observed in the sensors according to the tested embodiment of the invention (FIGS. 10c and 10d).

Crack Geometry

The inventors found that strain sensing performance could be programmed by adjusting crack depth, size and the shape of the localized cracking area.

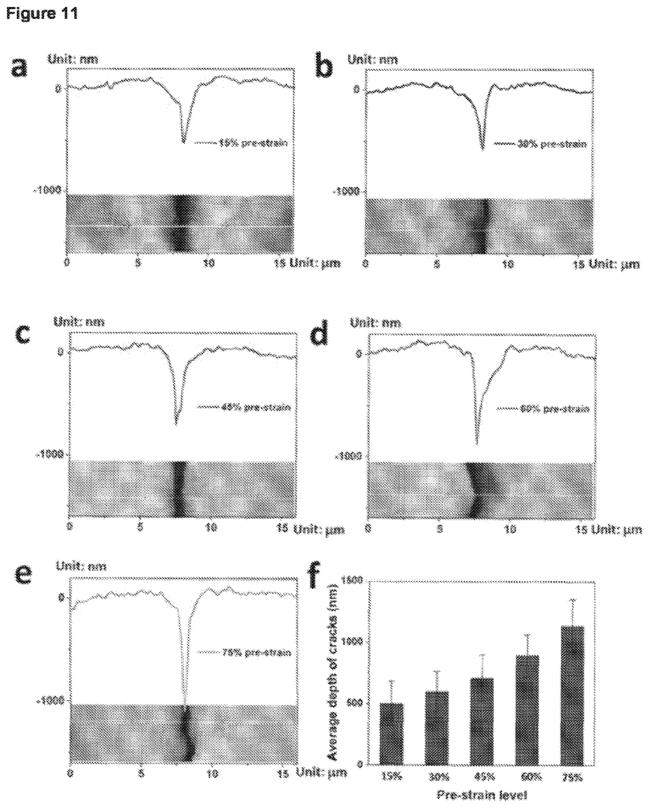

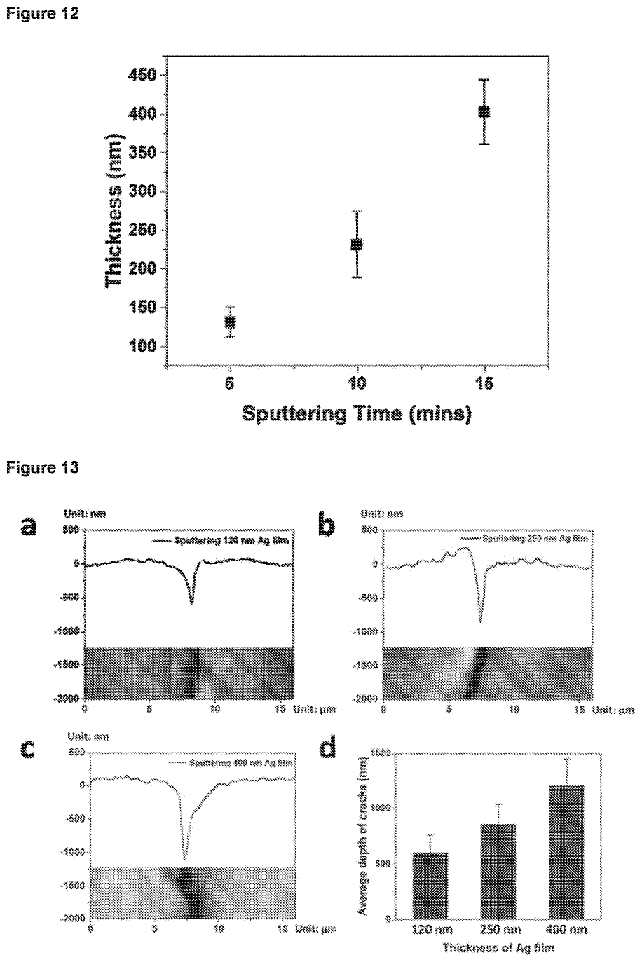

The crack depth may be controlled by adjusting the pre-strain level (pre-E) and/or the thickness (s) of the sacrificial metal layer following an approximately linear increase function (FIGS. 11-13).

As shown in FIG. 11, AuNWs/PDMS thin films with a Ag sacrificial metal layer deposited at a thickness of 120 nm, with pre-strain levels of 15%, 30%, 45%, 60% and 75% generated cracks of average depth of about 500 nm to about 1.2 μm.

As shown in FIG. 13, AuNWs/PDMS thin films with a Ag sacrificial metal layer deposited at a thickness of about 120 nm, 250 nm or 400 nm, with a pre-strain level of 30%, generated cracks of average depth of about 600 nm to about 1.2 μm.

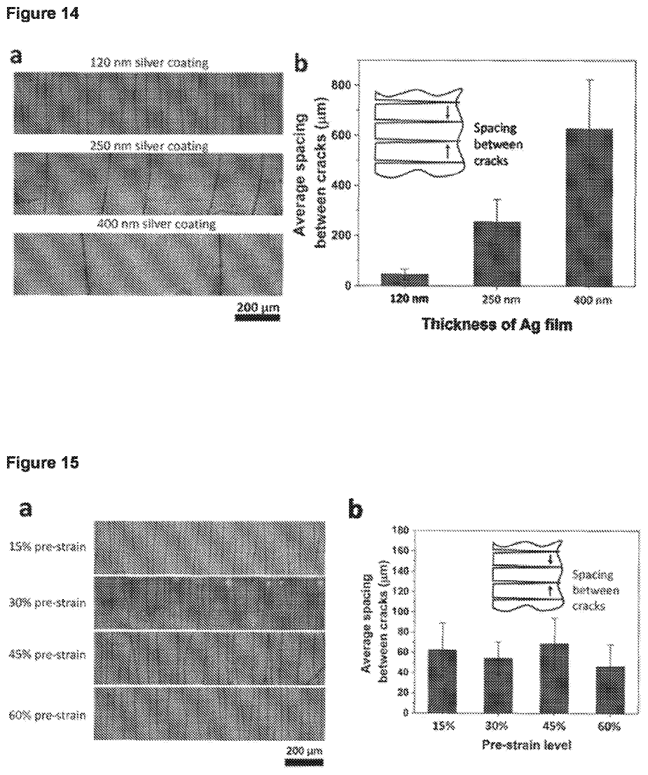

The crack spacing may be controlled by the thickness of the metal layer. As shown in FIGS. 14 and 15, the crack spacing decreased as the Ag metal layer thickness increased.

AuNWs/PDMS thin films with a Ag sacrificial metal layer deposited at a thickness of about 120 nm, 250 nm or 400 nm, with a pre-strain level of 30%, generated cracks with an average spacing of about 50 nm to about 600 nm (FIG. 14).

AuNWs/PDMS thin films with a Ag sacrificial metal layer deposited at a thickness of about 120 nm, with pre-strain levels of 15%, 30%, 45%, and 60%, generated cracks with an average spacing of about 45 nm to about 75 nm (FIG. 15).

Sensitivity and Stretchability

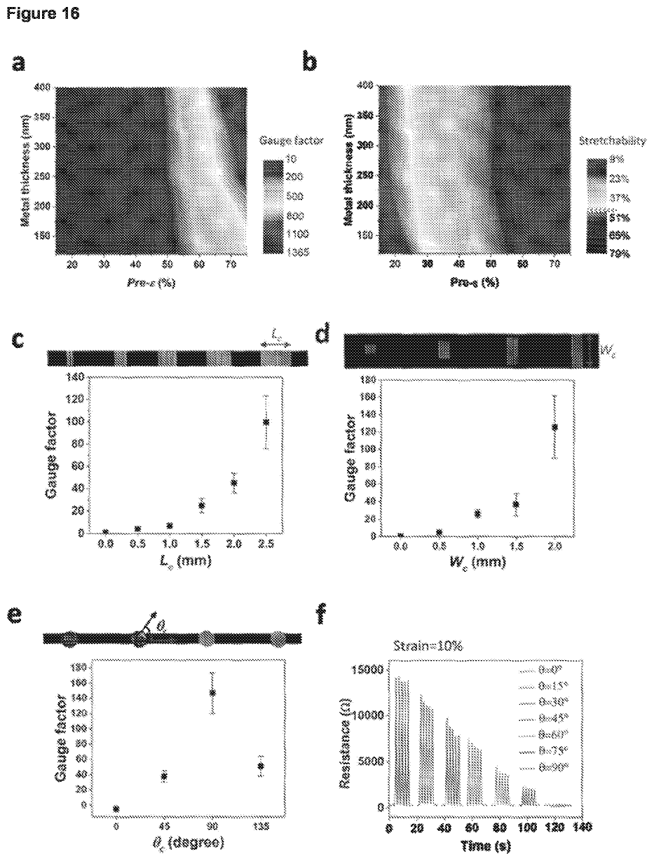

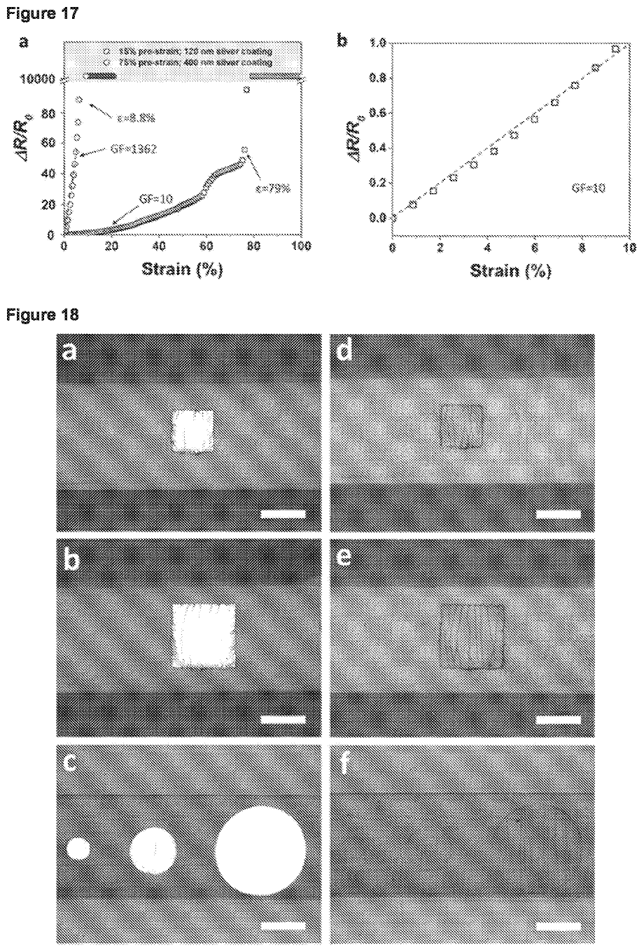

The inventors tested how sensitivity and stretchability limit (εₗᵢₘᵢₜ) affects the two parameters (pre-ε and s) in two-dimensional mapping graphs (FIGS. 16a and 16b). As used herein, sensitivity is defined as GF=ΔR/(R₀ε) in the strain range of 0-10%, where ΔR is the change in electrical resistance, R₀ is the initial electrical resistance, ε is the tensile strain; stretchability limit (εₗᵢₘᵢₜ) is defined as the value of tensile strain that the cracked sensor lost its conductivity. As shown in FIGS. 16a and 16b, the highest GF zone is triangular, located in the upper right corner of the GF map corresponding to the deepest crack depth zone, indicating both pre-strain level and metal thickness determines the sensitivity of cracked E-skin. In contrast, the optimal εₗᵢₘᵢₜ zone is rectangular, located on the left edge of stretchability map. It indicates pre-strain level rather than sacrificial metal thickness determines the εₗᵢₘᵢₜ value. The finely and widely tuning gauge factors (GFs) from 10 to 1360 in the typical strain range of 9%-79% (FIG. 17) is in contrast with previous reported rigid cracked system, which only allow a tunable GF in the high sensitivity range (GF>2,000) with very limited stretchability (εₗᵢₘᵢₜ<5%).

As shown in FIG. 18, the location and dimensions of the cracking area may be tuned simply by using different shadow masks.

Crack geometry, including length (Lc) and width (Wc), influence strain sensing performance as shown in FIGS. 16c and 16d. GF values are proportional to the length and depth of cracked area on the V-AuNWs strip. GF values increased from 1 to 99 when the length of the cracked area increased from 0 to 2.5 mm; and from 4 to 125 when the width of cracked area increased from 0.5mm to 2 mm (FIG. 19).

Crack Orientation

Controlling the orientation of cracks relative to the straining direction (θ_{c}), was found to have a direct implication on the sensing performance (FIG. 16e). A sensor with a crack orientation perpendicular to the strain direction generates a dramatic increase in resistance upon stretching (FIG. 19c, purple rhombus), whereas, a sensor with a crack orientation parallel to the strain direction only shows a slight resistance decrease in the same strain level (FIG. 19c, black square). Sensors with off-axis crack orientations of 45° and 135° to the strain direction display a moderate sensitivity in between in response to the same stretch.

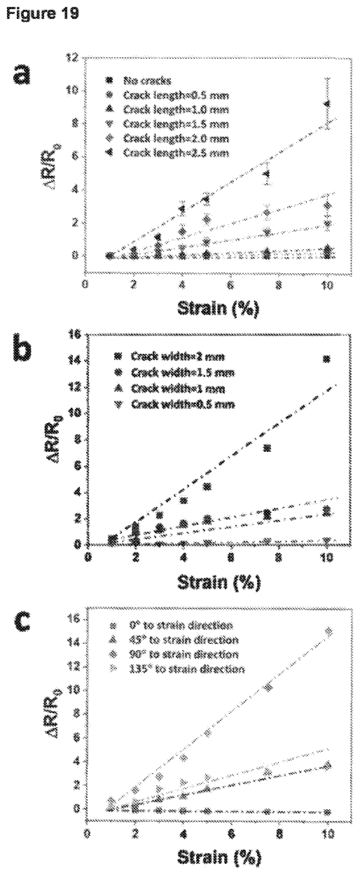

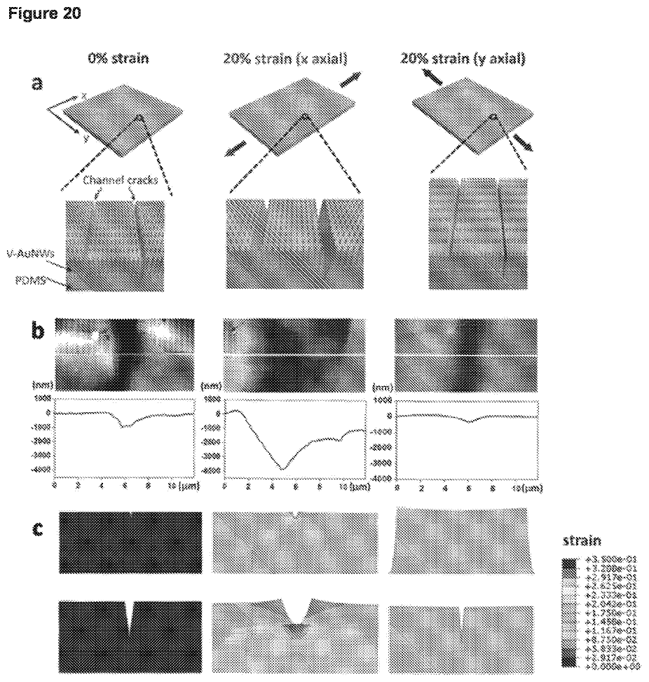

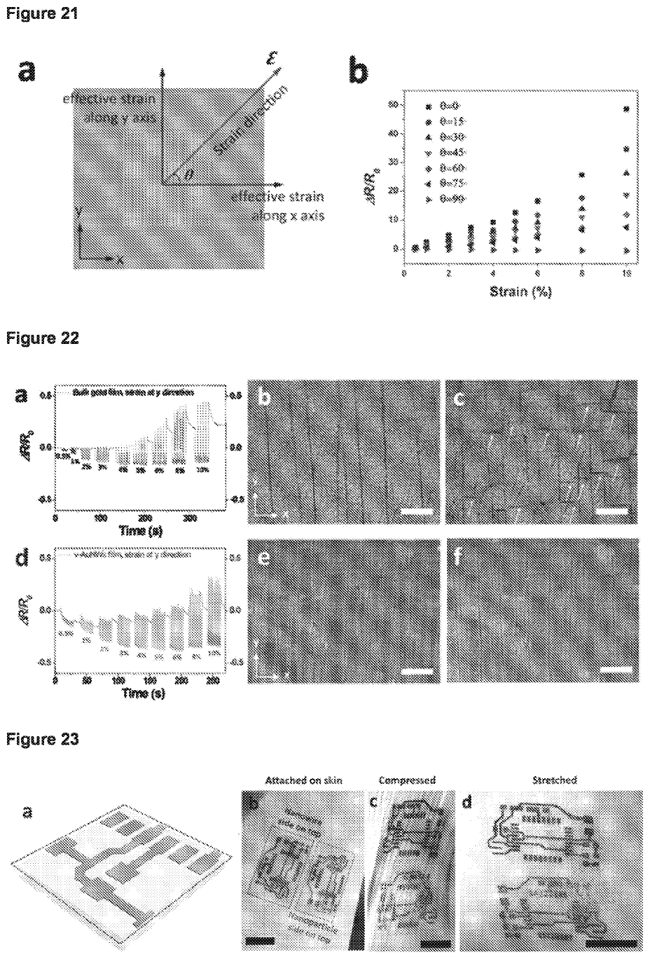

The anisotropic strain-directional sensitivity may be explained by the way that cracks respond to an external strain as shown in FIG. 20. The strain in the perpendicular direction enlarged the crack, hence, caused major change in conductivity. In contrast, the strain applied along the crack orientation reduce the crack due to Poisson's effect, leading to minimal changes in conductivity. The off-axis strain could be regarded as the combination of effective strain in both x and y axis, which showed a sensitivity highly dependent on the strain direction (FIG. 16f and FIG. 21). This phenomenon could also be observed in the conventional cracked bulk gold film (FIG. 22). However, additional cracks were generated when an off-axial strain was applied due to the poor adhesion between sputtered gold film with elastomeric PDMS, leading to permeant conductivity losses after strain release. In contrast, the cracked AuNWs/PDMS thin films exhibited no conductivity changes after strain release, as no additional cracks were generated.

Circuits

FIG. 23a depicts a schematic of a circuit structure including one or more sensors according to any embodiment of the first aspect of the invention. A AuNWs/PDMS thin film including at least one slit passing between neighbouring AuNWs serves as a sensor. The circuit includes one or more conduction paths for electrically connecting circuit elements. The conduction paths are AuNWs/PDMS thin films that do not include slits, and are suitable to serve as interconnects due to their strain-insensitive properties. As shown in FIG. 23, the sensors and conductive tracks share a common PDMS elastomeric body in the form of a membrane or sheet.

Because the conductive tracks and sensors are of the same composition (gold), the circuit structure advantageously eliminates the need for soldering or gluing the planar integrated multi-sensing circuit components.

The circuit depicted in FIG. 23 includes 5 sensors according to the first aspect of the invention in a complex gold circuit decal (4×5 mm²) printed on the human skin. This ultrathin gold circuit decal strongly adhered to skin naturally by van der Waals forces with excellent conformal contact during various skin deformation. The inventors were able to accurately extract biometric information by “stick-and-play” in a wireless manner via a Bluetooth Low Energy (BLE) module.

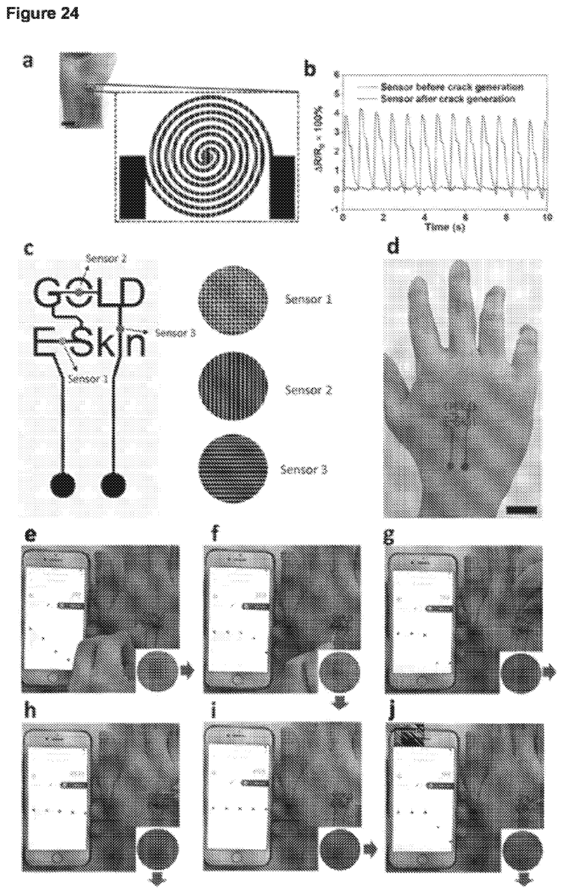

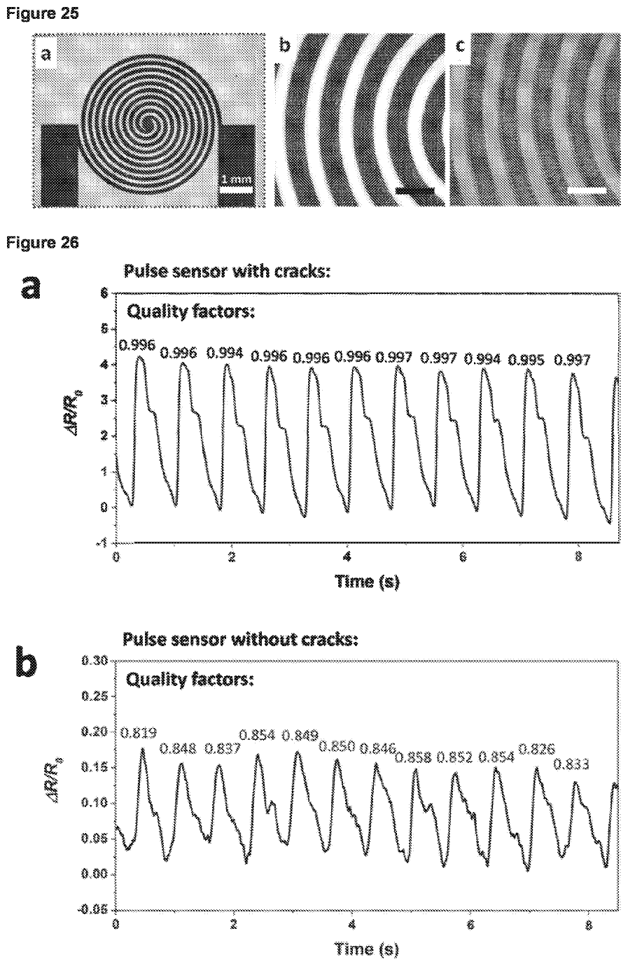

FIGS. 24 and 25 depict spiral shaped circuit structures. The circuit structures include a conductive track for electrically connecting circuit elements, wherein the conductive track comprises an AuNWs/PDMS thin film (FIG. 25b). Cracks were generated having an orientation transverse to the conductive track (FIGS. 25a and 25c).

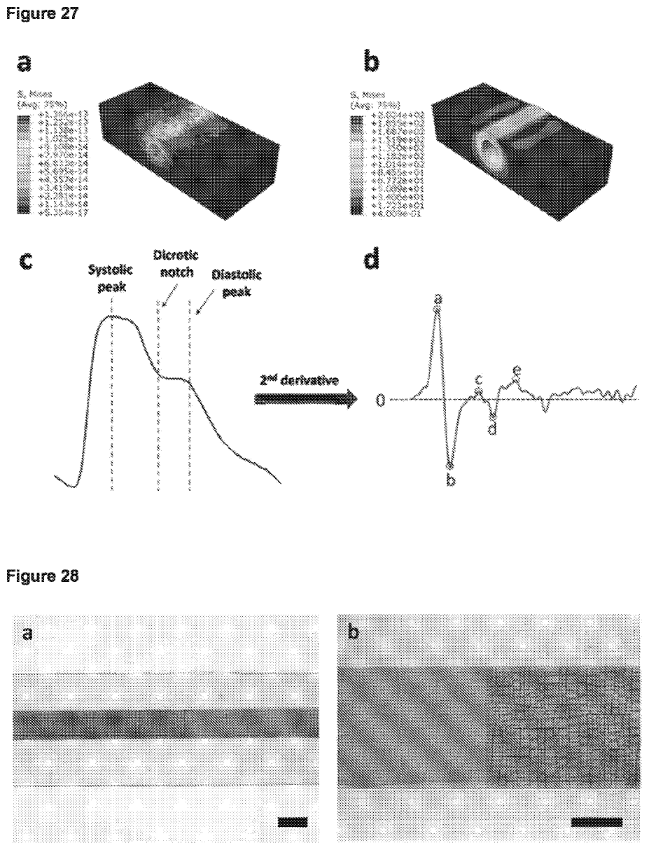

The spiral-shaped gold circuit decals were placed directly onto the radial artery wrist pulse area (FIG. 24a and FIG. 25) where specific blood vessel expansion/contraction was strongest. The quality of pulse signals from the cracked sensor was significantly improved compared to the non-cracked circuit structure (FIG. 24b). The quality of signals could be quantified by the Signal Quality Index (SQI) between 0 (lowest quality) and 1 (highest quality) (See details in FIG. 26). The average SQI value for the crack-based pulse sensor was 0.987, compared to 0.856 for the non-crack-based sensor. The second derivative of pulse waves is an indicator of the acceleration of the blood in arteries. From a series of waveforms measured with the crack-based spiral-shaped pulse sensor, the 2nd derivative wave (FIG. 27) agreed well with the literature data for a healthy 30-year-old male.

Multi-Sensing Circuits

FIG. 24 shows a multi-sensing circuit. Three specific direction-discrimination sensors in gold circuit decal were printed onto the dorsal aspect of hand (FIGS. 24c and 24d). The circuits were tested by simply sticking a Bluetooth Low Energy (BLE) module with the assist of a bandage patch, without the need of soldering or gluing. As seen from wireless signals recorded in a smartphone, a bi-directional cracked sensor (FIG. 28) was sensitive to strain in all directions, while vertical (sensor 2) and horizontal (sensor 3) cracked sensors were only sensitive to the directions perpendicular to the cracks (FIG. 24e-j).

As the largest organ, human skins are multifunctional yet specific. Different parts of human skins may experience multi-axial forces and undergo a range of angular and linear motions at specific locations, which require specific sensitivity and stretchability. To mimic this function, the inventors designed 11 crack-programmed specific sensors and 3 non-cracked sensors and integrated them within an area of 2.2×2.8 cm² in a planar layout (FIG. 29a). This design offered multi-sensing modalities of specific strains (I), specific pressures (II), strain directions (III), temperature (IV), glucose sensor (V), and lactate sensor (VI). In the strain zone I, from S1 to S4 strain sensitivity decreased but stretchability increased (FIG. 29b), which was realized by adjusting crack length; in the pressure zone II, from P1 to P4 pressure sensitivity decreased with the decrease of crack area size (FIG. 29c); in the strain direction zone III, local cracks were programmed in four different directions (D1-D4). The comparison of relative signal strengths could be used to identify particular strain directions (FIG. 29d).

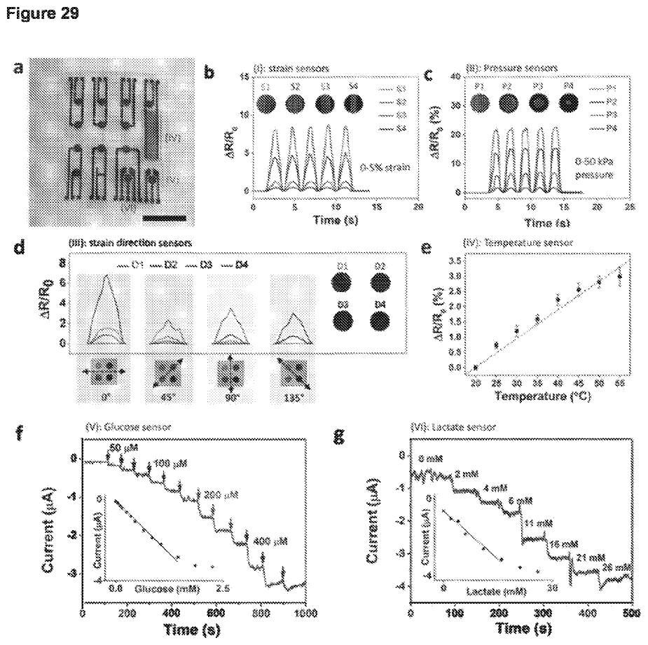

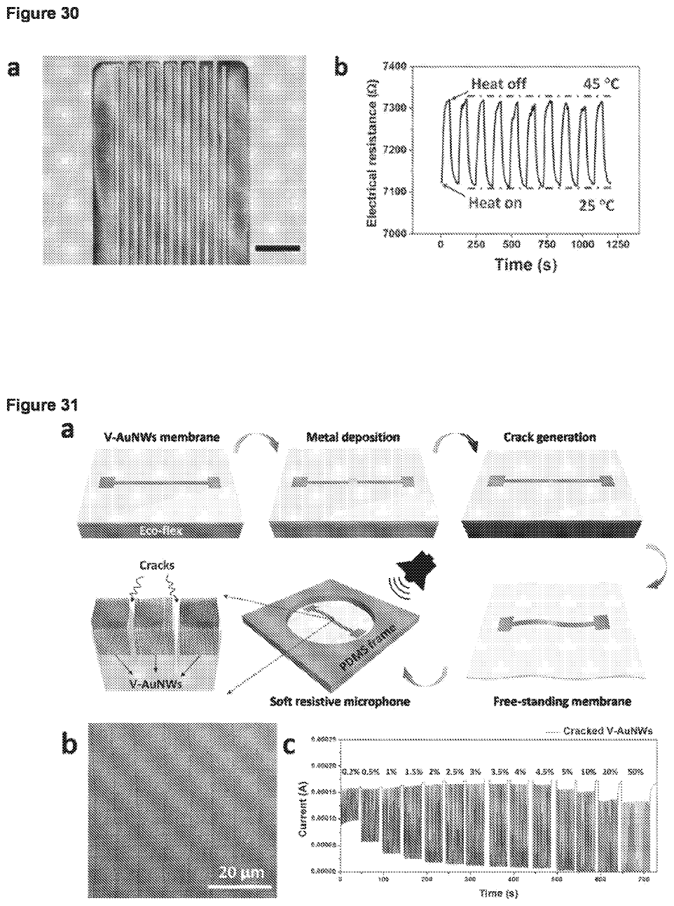

In the temperature zone IV, non-cracked serpentine pattern could serve as a resistive temperature sensor (FIG. 30). The relative resistance exhibited a linear response to the temperature level from 20 to 55° C. (FIG. 29e). The temperature coefficient of resistance (TCR) may be estimated by TCR=ΔR/(R₀ΔT), where R₀ is the original resistance value of the sensor and ΔR is the resistance change corresponding to the temperature change ΔT. The TCR of our sensor is 928 ppm/° C., comparable to the commercial products.

The non-cracked gold nanowire E-skins may serve as glucose and lactate sensors in a standard 3-electrode system, in which blank gold E-skin served as counter electrode, glucose oxidase- or lactate oxidase-modified gold E-skin served as working electrode and Ag/AgCl modified gold E-skin served as reference electrode. FIG. 29f shows the representative current responses of the glucose sensors, with staircase-like chronoamperometric curve. A linear fit led to an areal sensitivity of 41.3 μA mM⁻¹ cm² (inset of FIG. 29f). Similar staircase chronoamperometric curve was obtained for lactate sensor (FIG. 29g), with an areal sensitivity of 3.4 μA mM⁻¹ cm⁻² (inset of FIG. 29g). The results indicate the potential of our E-tattoos for real-time sweat glucose/lactate monitoring.

### EXAMPLES

Chemicals

Gold (III) chloride trihydrate (HAuCl₄.3H₂O, 99.9%), Triisopropylsilane (99%), 4-Mercaptobenzoic acid (MBA, 90%), (3-Aminopropyl)triethoxysilane (APTES), sodium citrate tribasic dihydrate (99.0%), L-ascorbic acid, polymethyl methacrylate, K₃Fe-(CN)₆, KCl, H₂O₂, HCl, liquid metal (EGaln), n-Hexane, acetone, and ethanol (analytical grade) were purchased from Sigma Aldrich. PDMS elastomer base and curing agent (Sylgard 184) were received from Dow Corning. Polymethyl methacrylate (PMMA), 950 A6, was purchased from MicroChem Corp. Positive photoresist AZ 1512 and developer AZ 726 MIF were received from Microchemicals GmbH. Bare silicon wafer <100> was purchased from ELECTRONICS AND MATERIALS CORPORATION LIMITED. All solutions were prepared using deionized water (resistivity >18 MΩ·cm−1). All chemicals were used as received unless otherwise indicated. Conductive wires were purchased from Adafruit.

Synthesis of Standing Gold Nanowires (V-AuNWs)

A modified seed-mediated approach was used, as described in the literature (Wang, Y. et al ACS Nano (2018) 12, 8717; Wang, Y. et al ACS Nano (2018) 12, 9742). Firstly, 2 nm gold seeds were synthesized. Briefly, 0.25 mL 25 mM ml Gold (III) chloride trihydrate and 0.147 mL 34 mM sodium citrate was added into a conical flask with 20 mL H₂O under vigorous stirring. After 1 min, 600 μL of ice-cold 0.1M NaBH₄ solution was added. The solution was then stirred for 5 min and stored at 4° C. until needed. To grow V-AuNWs on substrates (e.g. poly(methyl methacrylate) coated silicon wafer), O₂ plasma was applied for 5 minutes to render the surfaces hydrophilic. Then the substrates were functionalized with an amino group by immersion in a 5 mM APTES solution for 1 h. APTES-modified substrates were further immersed into citrate-stabilized Au seeds solution for 2 hours to ensure the saturated adsorption of gold seeds, followed by rinsing with water two times to remove excess seed particles. Finally, Au seed-anchored substrates were immersed in a growth solution containing 980 μM MBA, 12 mM HAuCl₄, 29 mM L-ascorbic acid for 3 minutes, leading to the formation of V-AuNWs thin films.

Fabrication of V-AuNWs Thin Film Incorporated in PDMS

The fabrication process is depicted in FIG. 1. At first a thin PMMA layer was spin-coated on a bare silicon wafer at 3000 rpm for 45 s and baked at 180° C. for 2 minutes. Subsequently, photoresist (AZ1512) was spin-coated on the wafer at 3000 rpm for 45 s, and patterns were formed via conventional photolithography and etching processes. After growth of V-AuNWs on the photoresist patterned poly(methyl methacrylate) (PMMA), PDMS base and curing agent were mixed (w/w=10:1) and spin-coated on the as-grown substrates at 500 rpm for 1 minute. After curing at 80° C. for 3 h, the PDMS was sectioned with a scalpel and peeled off from the substrate. The standing AuNWs patterns were transferred and embedded in PDMS film.

Fabrication of Programmable Cracks

The V-AuNWs/PDMS electrodes with different shapes was covered by a shadow mask, leaving the desired area exposed for gold or silver sputtering. A layer of gold (or silver) thin film was sputtered with speed of 0.3 nm/s. Localized channel cracks could be formed on V-AuNWs/PDMS after applying a repeated strain of for 10 cycles using a uniaxial moving stage (THORLABS Model LTS150/M). Gold thin film could be removed by tape, while silver thin film could be dissolved by hydrogen peroxide/ammonium hydroxide (1:1) solution.

Fabrication of Glucose and Lactate Sensors

2 μL 1 mg/mL carbon nanotube solution was firstly dip-casted on the surface of V-AuNWs/PDMS working electrode, Prussian blue (PB) was electrodeposited after CV scan from 0-0.5V with the scan rate of 0.02 V/s for 8 cycles in freshly made PB solution containing 2.5 mM FeCl₃, 2.5 mM K₃Fe(CN)₆, 0.1 M KCl and 0.1 M HCl. Then 2 μL 20 mg/mL glucose oxidase (GOx) or lactate oxidase (LOx) was drop-casted on the surface of working electrode for glucose sensor and lactate sensor, respectively. After drying at room temperature, 2 μL 1% chitosan solution in 2% acetic acid was dropped on the working electrode. Ag/AgCl ink was brush painted on the V-AuNWs reference electrode and baked at 100° C. for 30 minutes.

Vibration Sensing

In a further aspect the present invention provides a vibration sensor. In the illustrative embodiment the vibration sensor is useable as an acoustic sensor e.g. as a microphone, but may be used for other types of vibration sensing.

FIG. 31a illustrates the steps in a method for making a vibration sensor according to the present embodiment. First an ultrathin V-AuNWs membrane is formed in accordance with the methods described herein. Firstly, 2 nm gold seed were synthesized. Briefly, 0.25 mL 25 mM HAuCl₄.3H₂O and 0.147 mL 34 mM sodium citrate was added into conical flask with 20 mL H₂O under vigorous stirring. After 1 min, 600 μL of ice-cold 0.1M NaBH₄ solution was added. The solution was then stirred for 5 min and stored at 4° C. until needed. To grow V-AuNWs on substrates (e.g. PMMA coated silicon wafer), O₂ plasma was applied for 5 minutes to render the surfaces hydrophilic. Then the substrates were functionalized with an amino group by immersed in a 5 mM APTES solution for 1 h. The substrate was further immersed into 2 nm gold seed solution for 2 hours to ensure the saturated adsorption, followed by rinsing with water two times to remove excess seed particles. Finally, Au seed-anchored substrates were immersed in a growth solution containing 980 μM MBA, 12 mM HAuCl₄, 29 mM L-ascorbic acid, leading to the formation of V-AuNWs thin films. The thin PMMA layer was spin-coated on bare silicon wafer at 3000 rpm for 45 s and baked at 180° C. for 2 minutes. After synthesis of V-AuNWs on the PMMA surface following the procedure demonstrated above, photoresist (AZ1512) was spin-coated on top of nanowires at 3000 rpm for 45 s, and electrode patterns were formed via conventional photolithography and etching processes. PDMS (w/w base:curing agent=10:1) was diluted with n-Hexane (w/w PDMS:n-Hexane=1:3) and spin-coated on the top of patterned photoresist at 1000 rpm for 1 minute. After curing at 80° C. for 3 h, the ultrathin V-AuNWs/PDMS film was lifted-off the PMMA surface by sonicating the wafer in acetone solution for 30 s, which was finally fished up by a water-dissolvable Polyvinyl alcohol (PVA) supporting paper. Next, the ultrathin V-AuNWs membrane was transferred onto an Eco-flex elastomer with the assistance of a water dissolvable Polyvinyl alcohol (PVA) tape.

Next the ultrathin V-AuNWs/PDMS film was covered by a shadow mask, leaving the desired area exposed for silver sputtering. A layer of silver thin film was sputtered with speed of 0.3 nm/s.

In a third step localized slits were formed in the ultrathin V-AuNWs/PDMS film by applying a repeated strain of for 10 cycles using a uniaxial moving stage (THORLABS Model LTS150/M). As discussed above the slits were formed by cracking at the portion of the ultrathin V-AuNWs/PDMS film that was covered by silver in the sputtering step.

Next, the silver thin film was then dissolved by hydrogen peroxide/ammonium hydroxide (1:1) solution.

Next the ultrathin cracked V-AuNWs/PDMS film was suspended onto a carrier formed by a PDMS frame. The PDMS frame had a circular aperture through it defining a void that is spanned by the sensor such that ultrathin PDMS film can move in concert with applied vibrations.

Sensors made in this fashion were made the subject of various experiments to characterise the vibration sensor when used to detect audio signals propagated in air.

FIG. 31c illustrates a plot of current changes of an exemplary sensor with dynamic tensile strain ranging from 0.2%-50% over 10 stretching-releasing cycles at each strain level. As shown the sensor comprising a V-AuNW conductive strip showed dramatic resistance increase caused by the application of tensile strain as described above. The cracked area lost its conductivity completely after a tensile strain of more than 10%. However, its electrical conductance could be fully recovered, even after releasing from a strain of 50%. This high durability under high repeated strain test is in contrast with prior art sputter coated metallic channel crack system, which could only allow a limited stretchability of <10%.

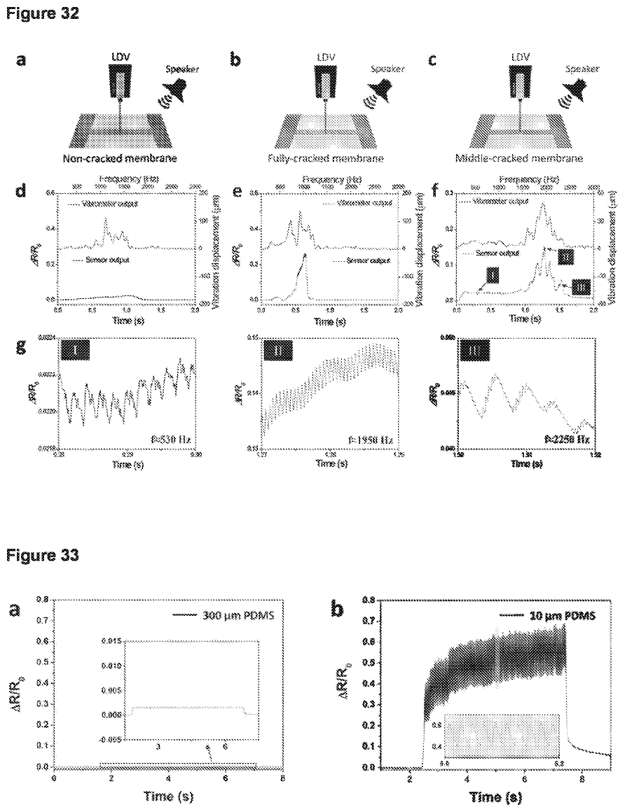

FIG. 32a-c are schematic illustrations of the experimental set-ups for acoustic measurement from a V-AuNWs/PDMS films with different slit set-ups. In particular the V-AuNWs/PDMS films were prepared in accordance with the parameters set out above, but with different slit layouts. In each case the membrane carrying the V-AuNWs/PDMS film mounted to a carrier as illustrated in FIG. 31a. The membrane of FIG. 32a has no slits formed in the V-AuNWs/PDMS film, whereas the sensor of FIG. 32b has slits the full length of the V-AuNWs/PDMS film spanning the void of the carrier, and a sensor according to FIG. 32c has localized slits (c) and is made as set out in connection with the slit formation process describe above in relation to FIG. 31a. As can be seen (and as will be apparent from the above description) the V-AuNWs/PDMS film produced very little sensor output, whereas the sensors of FIGS. 32b and 32c produced strong sensor outputs reflecting the variability in conductivity of the sensor in response to deformation caused by the sound waves.

However, it should be noted that the sensor output of the preferred form of the sensor FIG. 32c, with localised slits (i.e. slits only in a portion of the sensor spanning the void in the carrier) produced an output more closely resembling the laser Dopler vibrometer (LDV) output.

The non-cracked membrane FIG. 32a is not sensitive enough to effectively sense the acoustic signal. However The fully-cracked membrane FIG. 32b showed poor correlation with the raw acoustic signals. This is possibly because the acoustic strain level is not evenly distributed along the membrane strip or synchronized. In contrast, the local-cracked membrane exhibited resistive responses in excellent agreement with the raw signals from the laser Doppler vibrometer with the capability of resolving fine details. FIG. 32g shows an enlarged view of resistance changes with time in three different time zones (or frequency bands) indicated in FIG. 32f.

It was also shown that the response of such a sensor is dependent on the thickness of the V-AuNWs/PDMS film relative to its size. In particular as illustrated in FIG. 33, the ratio of ΔR to R₀ was low in a V-AuNWs acoustic sensor with a PDMS layer thickness of 300 μm (box a) and having dimensions of 10 mm×10 mm, compared to a V-AuNWs acoustic sensor with a PDMS layer thickness of 10 μm (box b) and having dimensions of 10 mm×10 mm. when a sound wave at constant frequency of 50 Hz was applied.

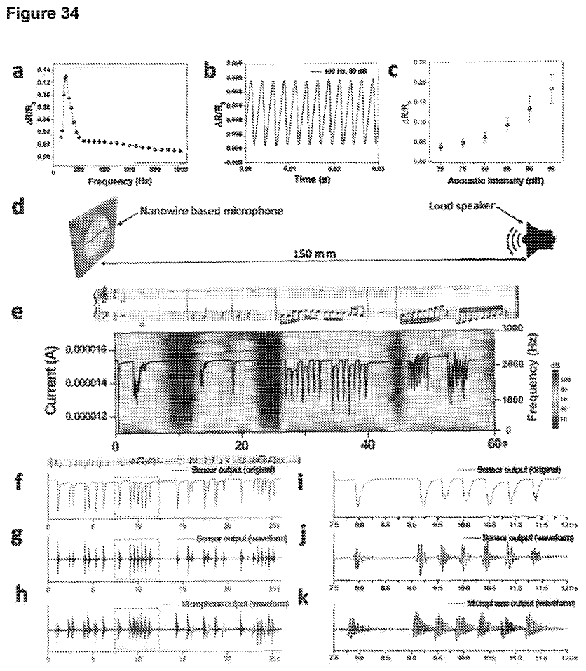

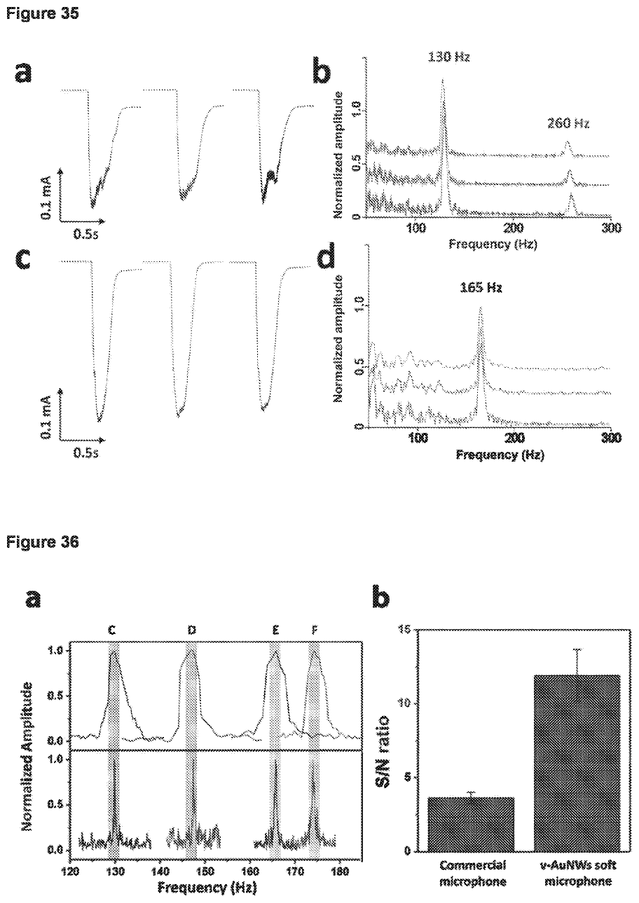

The inventors further monitored the electrical output from an exemplary acoustic sensor in response to sound coming from a speaker. Results of such testing is illustrated in FIGS. 34 and 35. FIG. 34d is a schematic of the experimental set-up of the music detection used to obtain the data of figures FIGS. 34a-34c, and 34e-34k.

FIG. 34a is a plot of the variation in resistance changes of an exemplary sensor as a function of sound frequency from 0-1000 Hz. The device exhibited stable and periodic electrical output across a frequency range between 50 Hz to 1000 Hz, with the maximum output being obtained at 80 Hz, after which it gradually decreased. FIG. 34b shows the detail of dynamic resistance changes of an exemplary sensor under sound with a constant frequency of 400 Hz at SPL of 90 dB.

The inventors also monitored the variations in the output resistance changes of the device with a decreasing sound pressure level (SPL) at a frequency of 80 Hz to determine its minimum sound-detection capability. FIG. 34c shows the variation in resistance changes of an exemplary sensor under sound with a constant frequency of 80 Hz at SPL from 70-95 dB.

The set up of FIG. 34d was also used to identify various music notes 150 mm away from a loud speaker. FIG. 34e shows the sensor output (curve) and the short time Fourier Transport (STFT) analysis (coloured background) in response to musical notes at different pitches. FIGS. 34(f) and 34(g) show the exemplary sensor output and waveform, compared to the conventional microphone output waveform in FIG. 34h in response to a piece of music. FIG. 34(i)-(k) show enlarged sections of the potions of the curves highlighted in dashed boxes in FIG. 34(f)-(h). The resistance changes of the sensor (curve in FIG. 34e) agreed very well with the spectrograms of the music sound (background in FIG. 34e).

Further analysis was performed by testing different notes from the loudspeaker, and performing a fast Fourier transform analysis to the sensor output. The results are shown in FIG. 35. FIG. 35a shows the original signal from the acoustic sensor for the detection of note “C” (C₃) repeated 3 times. FIG. 35b shows an FFT analysis of the sensor output from a. FIG. 35c represents the same test with an “E” (E₃) note repeated for 3 times, and FIG. 35d is the corresponding FFT analysis. As can be seen the notes are reproduced reliably on each test, and the FFTs display very similar frequency peaks and harmonics.

FIG. 36a shows the normalised output of a sensor in response to music notes for an exemplary audio sensor and commercial microphone. A piece of music with continuous notes was measured by the acoustic sensor and a commercial microphone simultaneously. The sensor of the embodiment of the present invention offered high-fidelity in detecting acoustic waveforms that is comparable to commercial microphone. Moreover the signal-to-noise ratio (FIG. 36b) was 4 times higher for the inventive embodiment compared to the commercial microphone.

Vibration Sensing in a Flexible Artificial Basilar Membrane

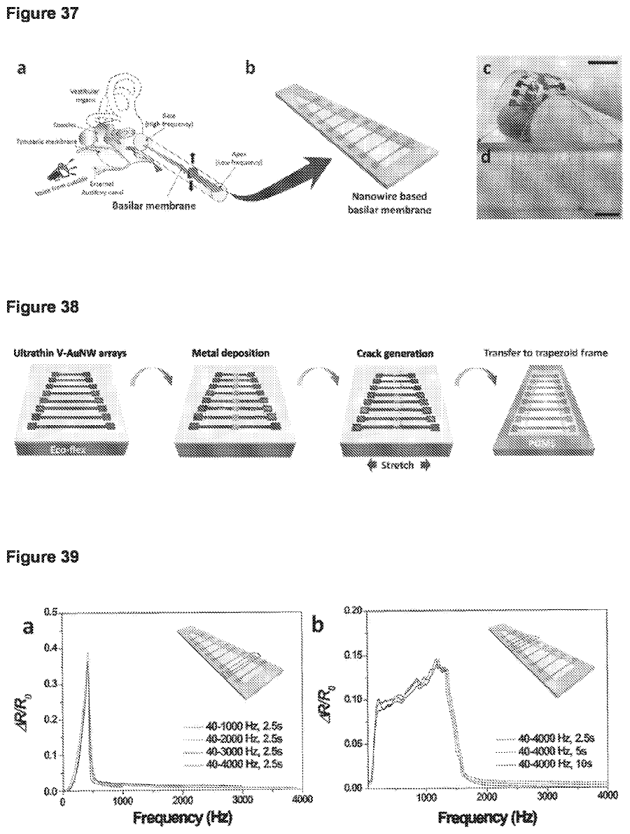

An embodiment of a vibration sensor will now be described which is applicable to use as an artificial basilar membrane (ABM). The present embodiment of a sensor offers a resistance based acoustic transducer that mimics the mechanical frequency selectivity of the human basilar membrane. FIG. 37a is a conceptual schematic of human ear with an uncoiled cochlea. In overview the exemplary ABM includes 8 nanowire-based sensors made according to an embodiment of the first aspect of the present invention. The sensors are formed as strips (as described above) and are mounted to a carrier in the form of a trapezoidal frame. FIG. 37b shows a schematic illustration of such a vibration sensor including 8 sensor strips with localized cracks in their center. FIG. 37c is a photograph of a soft nanowire-based artificial basilar membrane made in accordance with FIG. 37b (scale bar 1 cm), bent to show the flexibility of the completed structure. FIG. 37d is an optical image showing the cracked area of a sensor of the device of FIG. 37c (Scale bar: 200 μm). The exemplary ABM exhibits frequency selectivity in the range of 319-1951 Hz and sensitivity of 0.48-4.26 Pa⁻¹.

The fabrication process of nanowire based ABM is illustrated in FIG. 38. The process is similar to the acoustic sensor described above, excepted that the pattern of photoresist mask is different. In the current embodiment, the length of the sensor strips 1-8 varied from 2-5.5 mm, with a 0.5 mm interval for each sensor strip. They were placed at a constant spacing a pitch of 0.5 mm. The carrier for the sensor was a trapezoidally shaped PDMS frame. The frame has two longitudinally extending side portions that support the sensor on either side of a void in the centre of the frame. In a preferred form the frame is an elongate trapezoidal structure having a pair of laterally spaced apart support members defining the lateral sides of an elongate void between them. By positioning the membrane with the plurality of sensors across the carrier the support members carry a plurality of sensors therebetween. As can be seen the sensors are spaced along the support members. The support members are arranged such that the void between them has a varying width between along the ABM.

The process begins with synthesis of V-AuNWs on a PMMA surface following the procedure demonstrated above. Photoresist (AZ1512) is applied and patterned to create the 8 sensor shapes via a photolithography and etching process. PDMS was applied to a thickness of 10 μm to create an elastomeric body incorporating the V-AuNWs to form conduction paths in part of the body. The PDMS membrane was transferred onto an Eco-flex elastomer as noted above. This produced the device of initial state in FIG. 38.

In the next step, metal deposition was performed in a center portion of the V-AuNWs conduction paths strip to enable slit formation in the membrane at those locations. Metal deposition was performed by sputtering a layer of silver onto the specific location through shadow masks.

Next in the third panel, of FIG. 38 repeated tensile stretching was performed in the direction of the arrows shown. The location without metal coating does not generate any slits. The silver metalisation layer was dissolved by hydrogen peroxide/ammonium hydroxide solution, leaving localized parallel slits as described above.

The soft nanowire-based acoustic sensor array incorporated into the PDMS membrane was transferred to a trapezoidal PDMS frame of the form described above.

Operation of the ABM created was characterized by application of sound and comparison with a laser Doppler vibrometer (LDV), as illustrated in FIGS. 39-41.

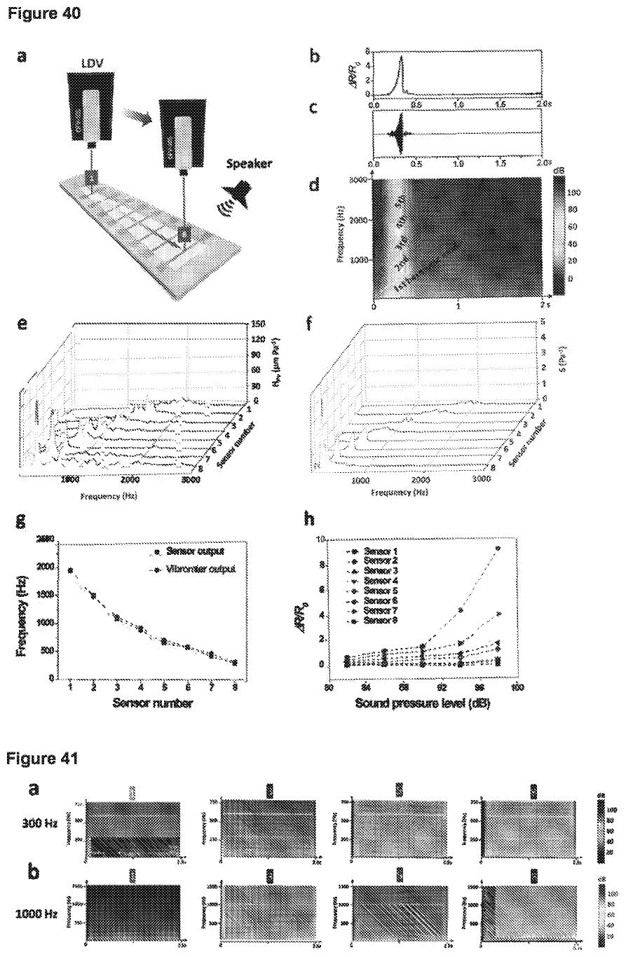

FIG. 40a is a schematic illustration of the experiment set-up for testing the sensor of FIG. 37b. In the results that follow the sensors forming the ABM are numbered from “1” starting at the shortest sensor (i.e. the sensor with the shortest free length) to “8” being the longest sensor. As illustrated in FIG. 40a, a speaker was used to produce a chirp sound in the frequency range of 40-3000 Hz for a duration of 2 s with approximately 82-88 dB of sound pressure. The mechanical vibration of a specific spot was sensed using a scanning laser Doppler vibrometer (LDV).

FIGS. 40b to 40d illustrates the sensor resistance response (b), sensor waveform (c), and short-time Fourier transform signals of a sensor strip under a chirp signal varying from 40-3000 Hz over for 2 s. The peak response in the Fourier transform signal (FIG. 40d) is at 575 Hz, reflecting the resonance frequency of the first harmonic mode in the middle of this sensor. The harmonic series (2^{nd}, 3^{rd}, 4^{th}, 5^{th} . . . harmonic modes) of this fundamental frequency are also observable in the sensor output of FIG. 40d. However, as the harmonic mode increases, the vibration amplitude is weakened.

In FIG. 40e acoustic-to-mechanical energy transfer function of each sensor is plotted for the same chirp. The measured mechanical displacement of the nanowire-based ABM was normalized by applied sound pressure. The acoustic-to-mechanical energy transfer function could be defined as H_{PD}:⁹

$${H}_{P D} ( f ) = \frac{D ( f )}{P ( f )} \qquad ( 1 )$$

where P is the sound pressure, D is the mechanical displacement at the geometric centre of the sensor strip, and f is the frequency of sound. Waterfall plots of H_{PD} for all sensor strips are shown in FIG. 40e. The peaks of the H_{PD} represent the resonance frequencies of the first bending mode for geometric centre of each sensor. Similar to human cochlea, the resonance frequencies of the transfer functions sequentially shift to higher frequencies with the increase of sensor strip length. In addition, the sensitivity (S) for acoustic sensors can be calculated by the equation (2):

$$S = \frac{ΔR / {R}_{0}}{P} = \frac{( R - {R}_{0} ) / {R}_{0}}{{P}_{0} \times 1 {0}^{{L}_{p / 20}}} \qquad ( 2 )$$

where R and R₀ are resistance of sensor before and after acoustic vibration, respectively. P₀ is the reference sound pressure of 0.00002 Pa and Lₚ is the sound pressure level in decibel. The measured sensitivity of each sensor strips is plotted in FIG. 40f.

The sensitivities of nanowire-based ABM sensors at the resonance frequencies were in the range of 0.48-4.26 Pa⁻¹, which is much higher than most prior art resistive wearable pressure sensors (which typically range from 0.00026 Pa⁻¹-0.606 Pa⁻¹ in the pressure range of 0-6 kPa).

The resonance frequencies measured from the sensitivity outputs match well with the transfer function (H_{PD}), with maximum error less than 10% (FIG. 40g).

In addition, the frequency range of the exemplary nanowire-based ABM falls in the human communication frequency range (300-3500 Hz), which can be directly used for speech recognition. FIGS. 39a and 39b also demonstrate that the resonance frequency, measured by peak value of sensor resistance change, is independent of the frequency range of the sound applied, and the time range of input of the chirp sound, supporting the conclusion that the resonance frequency detected by our ABM is valid.

To identify the dynamic range of electrical outputs of the exemplary sensors of the ABM with application of acoustic stimulus with different sound pressure levels, a pure-tone was applied to each sensor at its resonance frequency (FIG. 40h). A highly distinguished signal is detected in the sound pressure range of 82-98 dB SPL.

The performance of the exemplary ABM is further verified by application of a tone of a constant frequency of 300 Hz and 1000 Hz, respectively. As shown in FIG. 41, sensor 8 exhibited the highest intensity upon 300 Hz pure tone, while the highest response towards 1000 Hz pure tone shifted to sensor 3. These results are consistent with their resonance frequencies as shown in FIG. 40e.

Characterization

Scanning electron microscopy (SEM) images were characterized using FEI Helios Nanolab 600 FIB-SEM operating at a voltage of 5 kV. Atomic force microscopy (AFM) was characterized by the Dimension Icon AFM using tapping mode. To test the electro-mechanical responses of V-AuNWs/PDMS strain sensors, the two ends of the samples were attached to motorized moving stages (THORLABS Model LTS150/M). Uniform stretching cycles were applied by a computer-based user interface (Thorlabs APT user), while the current changes were measured by a VERSASTAT 3-500 electrochemical system (Princeton Applied Research). The performance of pressure sensor was done using SmarAct stepping positioner (SLC-1730) controlled by custom LabView program and force data measured by a GSO series load cell with capacity of 25 g (GSO-25). connected to Keithley 2604B SourceMeter. The electrical properties were measured simultaneously using two probe method with Keithley 2604B SourceMeter with a computer-based user interface. For temperature sensing, the sensor was fixed near a hot plate with adjustable temperature. The surface temperature was recorded by a portable infrared temperature detector. The CV and chronoamperometry of glucose and lactate sensor were measured by the VERSASTAT 3-500 electrochemical system (Princeton Applied Research). For the acoustic sensing, a high sampling rate of 10,000 was set to measure the current changes of samples with a constant voltage of 0.1V. For the experiment set-ups of acoustic sensing, a loudspeaker was located beside (direction of loudspeaker and ABM are kept at 45°) the sensor to produce sound. A Compact Digital Sound Level Meter (Jaycar, QM1589) was fixed near the acoustic sensors and ABM to measure the SPL around the device. An LDV system (OFV-2570, Polytec) was positioned perpendicular to the geometric centre of sensor strip along the longitudinal direction, thus measuring the displacement of each point upon application of the chirp sound. The chirp sound was produced by Labview at sampling rate of 10,000. For the music notes sensing, a commercial microphone was located near our nanowire-based acoustic sensor, which captured the sound emitted by the loudspeaker.

1. A sensor comprising an elastomeric body, said elastomeric body incorporating a plurality of discrete electrical conductors therein such that an electrically conductive path can be formed within the elastomeric body via conduction between neighbouring conductors, said elastomeric body including at least one slit passing between neighbouring conductors.

2. The sensor according to claim 1, wherein deformation of the elastomeric body changes a separation between neighbouring conductors having a slit therebetween to thereby change the conductivity of the electrically conductive path within the elastomeric body.

3. The sensor according to claim 1, wherein the discrete electrical conductors within the elastomeric body are nanowires.

4. The sensor according to claim 1, wherein the elastomeric body comprises a plurality of slits.

5. A circuit structure including one or more sensors according to claim 1.

6. The circuit structure according to claim 5, wherein the one or more sensors share a common elastomeric body.

7. The circuit structure according to claim 6, further including at least one of:
one or more further sensors; and
one or more conductive tracks for electrically connecting other circuit elements.

8. A decal comprising
a first layer including at least one sensor according to claim 1; and
a substrate.

9. The decal according to claim 8, wherein, in the first layer, the at least one sensor comprises a circuit structure according to claim 5.
